(19) United States
(12) Patent Application Publication
(10) Pub. No.: US 2010/0323015 A1
(43) Pub. Date: Dec. 23, 2010

(54) MODIFIED RELEASE FORMULATION AND METHODS OF USE

(76) Inventor: Biljana Nadjsombati, Irvine, CA (US)

Correspondence Address: MCDERMOTT, WILL & EMERY LLP, 600 13th Street, NW, Washington, DC, 20005-3096, US

(21) Appl. No.: 12/690,889

(22) Filed: Jan. 20, 2010

Related U.S. Application Data
(63) Continuation-in-part of application No. 12/505,409, filed on Jul. 17, 2009.
(60) Provisional application No. 61/082,162, filed on Jul. 18, 2008.

(51) Int. Cl. A61K 31/27; A61K 9/00; A61P 25/08

(52) U.S. Cl. 424/487; 514/485

(57) ABSTRACT

A modified release pharmaceutical formulation includes about 30-70% N-(2-amino-4-(fluorobenzylamino)-phenyl) carbamic acid ethyl ester (retigabine), or a pharmaceutically acceptable salt, solvate or hydrate thereof, about 5-30% of a drug delivery matrix including hydroxypropylmethylcellulose (HPMC), and an enteric polymer. The pharmaceutical formulation produces a sustained plasma concentration of retigabine following administration to a subject for 4-20 hours longer than the time required for in vitro release of 80% of retigabine. The plasma concentration vs. time profile of this formulation is substantially flat over an extended period lasting for about 4 hours to about 36 hours. A method of treating a disorder characterized by nervous system hyperexcitability includes administering to a subject an effective amount of these pharmaceutical formulations.

### STATEMENT OF RELATED APPLICATIONS

This application is a continuation-in-part of, and claims the benefit of priority to, U.S. patent application Ser. No. 12/505,409, filed Jul. 17, 2009, which in turn claims priority to U.S. provisional application No. 61/082,162 filed Jul. 18, 2008, each of these documents are incorporated herein by reference in their entirety.

### BACKGROUND OF THE INVENTION

This invention relates generally to pharmaceutical compositions and, more specifically to pharmaceutical formulations for the efficacious treatment of nervous system hyperexcitability.

Many solid oral pharmaceuticals such as tablets or capsules are formulated such that the active ingredient is immediately released upon administration. Generally, such immediate release (IR) dosage forms result in an initial very high blood level concentration that is followed by a rapid decline. One potential result of an immediate release dosage form is that the patient experiences varying degrees of blood level fluctuation, which can result in transient therapeutic overloads, followed by a period of therapeutic under-dosing. These blood level fluctuations, or peaks and troughs, are difficult to regulate and lower the overall therapeutic benefit of the administered dose.

Many immediate release oral dosage forms are administered more than twice a day to maintain a therapeutic level of active ingredient within these blood level fluctuations. However, multiple dosing does not alleviate the fluctuations, but merely reduces the degree or duration of either or both overload and under-dosing. Moreover, the more than twice daily dosing also can result in a poor patient compliance.

Delayed or controlled release formulations also have been developed for a number of active ingredients. However, such delayed release formulations exhibit disadvantages which affect their suitability to a particular drug or therapeutic objective. Moreover, these types of formulations are generally designed to delay the release of active ingredient in an effort to dampen the extent of dose overloads and under dosing. However, once released the active ingredient can still exhibit fluctuations in blood level concentrations.

Thus, there exists a need for a reliable formulation that delivers relatively constant levels of active ingredient over a sustained period of time. The present invention satisfies this need and provides related advantages as well.

### SUMMARY OF INVENTION

In some aspects, embodiments of the present invention relate to a modified release pharmaceutical formulation that includes about 30-70% N-(2-amino-4-(fluorobenzylamino)-phenyl) carbamic acid ethyl ester (retigabine), or a pharmaceutically acceptable salt, solvate or hydrate thereof, about 5-30% of a drug delivery matrix including hydroxypropylmethylcellulose (HPMC) and an enteric polymer. The pharmaceutical formulation produces a sustained plasma concentration of retigabine following administration to a subject for 4-20 hours longer than the time required for in vitro release of 80% of retigabine.

In other aspects, embodiments of the present invention relates to a formulation that includes about 30-70% N-(2-amino-4-(fluorobenzylamino)-phenyl) carbamic acid ethyl ester (retigabine), or a pharmaceutically acceptable salt, solvate or hydrate thereof, about 5-30% of a drug delivery matrix, and an agent for retarding release in the gastric environment. The plasma concentration vs. time profile of this formulation is substantially flat over an extended period lasting for about 4 hours to about 36 hours.

In still other aspects, embodiments of the present invention relate to a method of treating a disorder characterized by nervous system hyperexcitability that includes administering to a subject an effective amount of these pharmaceutical formulations.

### BRIEF DESCRIPTION OF THE DRAWINGS

FIG. 1 compares the in vitro dissolution and in vivo absorption profiles for the delayed release formulations for Avinza® and Kapinol® (Kadian®). FIG. 1A shows the dissolution profiles for Avinza® and Kapinol® under simulated intestinal fluid. FIG. 1B shows the plasma concentration for Avinza® and Kapinol® following administration to a subject.

FIG. 2 shows a comparison of the retigabine concentration-time profile simulated based on dissolution results with that of observed retigabine concentration-time profiles following administration in a modified release formulation of the invention.

FIG. 3 shows pharmacokinetic concentration-time profiles of exemplary formulations in healthy subjects under fed and/or fasted condition compared to an immediate release formulations or to a control formulation.

FIG. 4 shows dissolution time profiles of retigabine for Formulations 1-9. Dissolution profiles of retigabine immediate release and in several formulations under simulated in vivo conditions in 0.1N HCl for 1 hour followed by Borate buffer (pH 7.5) for 4-5 hours.

FIG. 5 shows the solubility of retigabine as a function of pH.

FIG. 6 shows mean concentration time profiles (dose normalized to 400 mg) of retigabine following administration of retigabine IR (400 mg) and retigabine modified release (MR) Example VIII (480 mg) and Example IX (480 mg) in the fasted state.

FIG. 7 shows mean concentration time profiles (dose normalized to 400 mg) of retigabine following administration of retigabine IR (400 mg) and retigabine MR Example VIII (480 mg) and Example IX (480 mg) in the fed state (High Fat).

FIG. 8 shows dissolution profile of Examples VIII and IX.

### DETAILED DESCRIPTION OF THE INVENTION

This invention is directed to pharmaceutical compositions having modified released properties of the active pharmaceutical ingredient retigabine. The modified release compositions of the invention result in a sustained plasma concentration of an active pharmaceutical ingredient for up to 20 hours or more. The modified release compositions of the invention are particularly useful for treatment of a wide variety of neurological-related disorders because sustained or prolonged plasma concentrations provide longer periods of pharmacological action. The benefits that can be realized due to these properties include enhanced efficacy, reduced dosages and decreased administrations. These and other characteristics also can lead to improved patient compliance and decreased incidences of adverse drug reactions.

In one specific embodiment, the invention is directed to a pharmaceutical composition containing the active pharmaceutical ingredient N-(2-amino-4-(4-fluorobenzylamino)-phenyl) carbamic acid ethyl ester or 2-amino-4-(4-fluorobenzylamino)-1-ethoxycarbonylaminobenzene. Exemplary formulation components for this specific embodiment can include about 10-15% drug delivery matrix, about 20-30% microcrystalline cellulose modified release polymer/binder, about 1-5% hypromellose 2910 modified release polymer/binder, about 3-5% copovidone binder, about 1% crospovidone disintegrant, about 2-7% croscarmellose sodium disintegrant, about 2-6% sodium dodecyl sulfate (SDS) surfactant, about 2-6% other surfactants, about 0.2-1.0% magnesium stearate lubricant, about 0.2-1.0% silicon dioxide glidant, and an enteric coating. Exemplary plasma concentrations can reach a maximum after about 10 hours or longer following administration and are sustained for about 10-20 hours or more. Beneficial plasma concentrations also can be observed for 30-40 hours post administration. The modified release pharmaceutical compositions containing 2-amino-4-(-fluorobenzylamino)-1-ethoxycarbonylaminobenzene are useful for treating a variety of disorders characterized by nervous system hyperexcitability and/or smooth muscle hyperexcitability, including seizure disorders such as epilepsy, neuropathic pain, inflammation, overactive bladder, urinary incontinence, functional bowel disorders, ulcerative conditions of the intestinal tract, hyperactive gastric motility, asthma, hypertension, migraine, and eating disorders. Generally, the modified release pharmaceutical compositions containing 2-amino-4-(-fluorobenzylamino)-1-ethoxycarbonylaminobenzene are useful as antidystonics, effectively reducing muscle tonicity and spasms. Additionally, these modified release compositions are useful as neuroprotective agents, for example, under conditions of reduced cerebral blood flow, such as during a stroke and other ischemia-related events, and for the treatment of vascular diseases affecting blood flow such as Raynaud's syndrome, impotence, premature ejaculation, female anoryasmia, clitoral erectile insufficiency, vaginal engorgement, dyspareunia and vaginismus. Additionally, the modified release composition is useful for achieving reversible cardiac arrest and restoring coronary blood flow. The modified release pharmaceutical composition is also useful for the treatment of neurodegeneration. Other disorders that are effectively treated by the modified release compositions include intermittent claudication, pollakiuria, nocturia, hyperreflexia, enuresis, alopecia, dysmenorrheal, begnign prostatic hyperplasia, premature labor, disorders associated with diabetes, such as retinopathy, neuropathy, nephropathy, peripheral circulation disorder, and skin ulceration. The modified compositions are also useful for treating behavioral disorders such as nicotine addiction withdrawal, mania, bipolar disease, and anxiety disorders.

The modified release compositions of the invention exhibit properties unlike those of typical slow release or delayed release formulations. Generally, slow or delayed release formulations are based on delaying the rate of dissolution or release of active pharmaceutical ingredient (API) so as to retard delivery of portions of or the entire dose. The in vivo adsorption profile of the API therefore parallels its in vitro dissolution profile. For example, if a slow release formulation releases an API over a 10 hour period, its absorption profile similarly will show an increasing or sustained plasma concentration over this 10 hour period, followed by a steady decline after release of most of the dose.

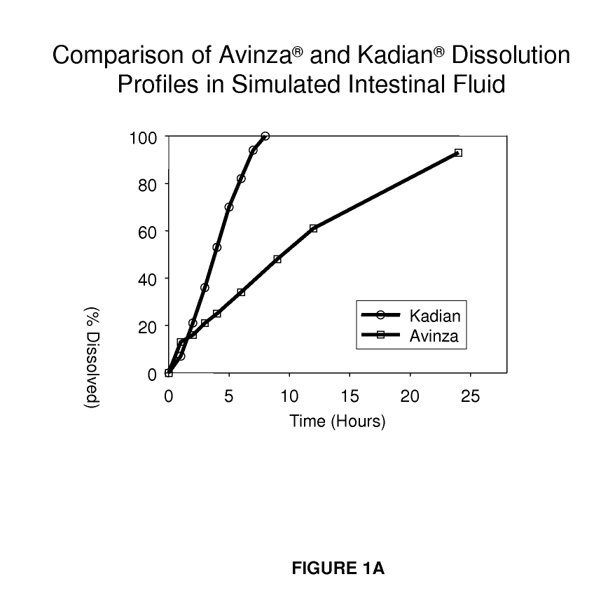

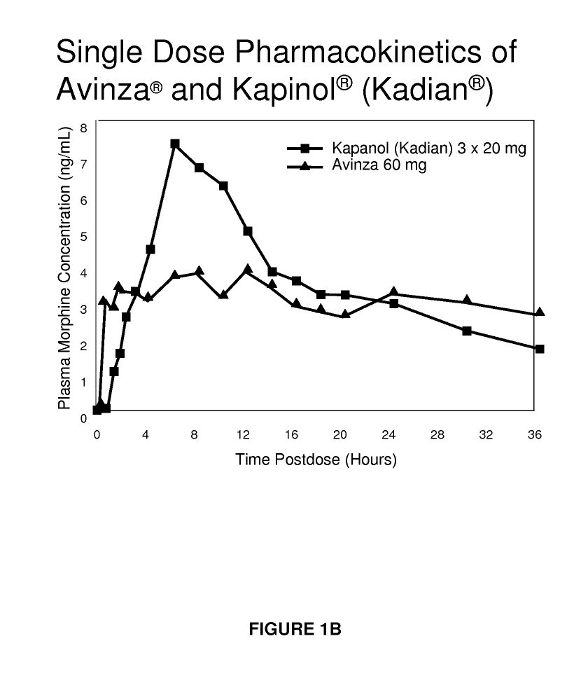

FIG. 1 exemplifies these slow and/or delayed release formulation properties for the two morphine formulations Avinza® and Kapinol® (Kadian®). FIG. 1A shows that the in vitro dissolution of Kadian® is about 100% complete at about 7 hours under conditions that simulate intestinal fluid (e.g., pH 7.5). The in vitro dissolution of Avinza® under these conditions is about 90% complete after about 24 hours. Correspondingly, the in vivo adsorption profiles parallel these delayed release rates. Kadian® plasma concentrations peak at about 6-7 hours post administration followed by a marked decline thereafter. Avinza® plasma concentrations exhibit a concentration profile that has a much lower maximum value, that is relatively constant over the duration of the 24 hour release period, and is followed by a decline thereafter.

In some embodiments, the modified release formulations of the invention exhibit very different in vivo absorption characteristics compared to what would be expected based on their in vitro dissolution profiles under simulated intestinal conditions. As described further below, the modified release formulations result in a steady release of retigabine where about 80% or more becomes dissolved by about 4-6 hours under simulated intestinal conditions. However, the in vivo absorption profiles as measured by retigabine plasma concentrations do not parallel the dissolution profiles. Rather, maximum retigabine concentrations are observed well after its peak release and are maintained at a significant plasma level for at least about 4-8 times longer than would be expected.

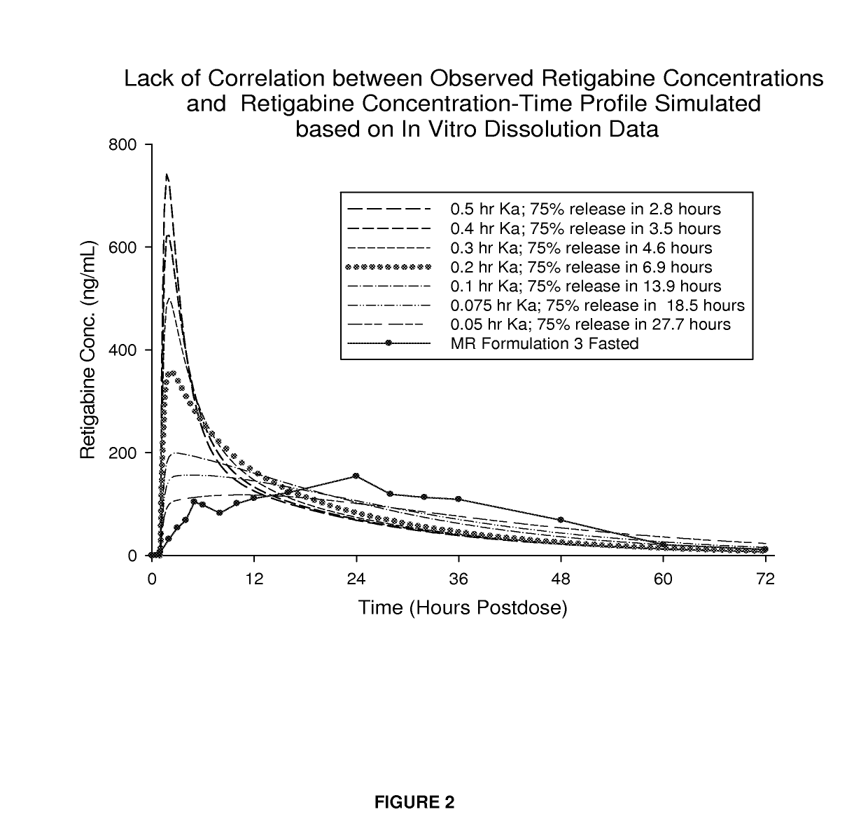

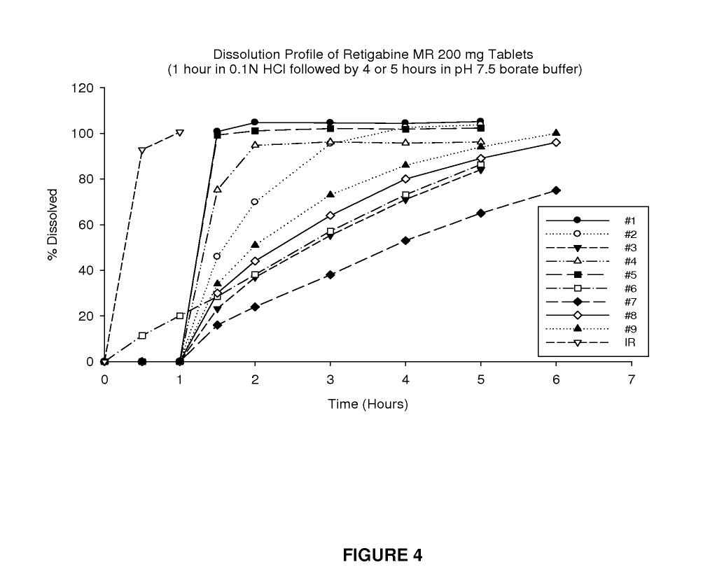

The lack of a correlation between expected and observed retigabine plasma concentrations is shown in FIG. 2. Briefly, FIG. 2 provides a simulation illustrating the effect of a change in the absorption rate constant (Kₐ), mimicking a change in the rate of retigabine dissolution, over a range of times that allows for 75% release and absorption of retigabine up to approximately 27 hours. This simulation included a lag of 1 hour to account for the inclusion of an enteric polymer as part of a coating on a modified release formulation of the invention. Release of 75% of the active ingredient by 6.9 hours, as provided by an Kₐ equal to 0.2 (dotted line), therefore represents a total of 7.9 hours following administration to a subject. This rate closely resembles observed in vitro dissolution results shown in FIG. 4 and Example V below.

Overlaid onto the above simulated changes in retigabine absorption is the observed absorption as illustrated in a concentration-time profile (circles ()) of an exemplary modified release formulation of the invention. The overlay of observed results shows a sustained absorption profile that achieves maximum concentration at about 24 hours after administration, or more than 18 hours post in vitro dissolution. These results indicate that the modified release formulations of the invention exhibit uncharacteristically long, sustained absorption based on their relatively quick dissolution properties. These modified release properties are particularly useful for delivering safe, efficacious doses of retigabine for the treatment of a wide variety of neuropathic disorders, including seizures and neuropathic pain as well as those exemplified previously.

An active pharmaceutical ingredient, or API or active ingredient refers to the chemical or substance in a drug that is pharmaceutically active. These terms are used herein synonymously and include all such art recognized meanings. An active pharmaceutical ingredient of the invention includes pharmaceutically acceptable forms of the chemical or substance. A specific example of an active pharmaceutical ingredient useful in the formulations of the invention is N-(2-amino-4-(4-fluorobenzylamino)-phenyl) carbamic acid ethyl ester or 2-amino-4-(4-fluorobenzylamino)-1-ethoxycarbonylaminobenzene. This compound also is known in the art as retigabine and has the structure:

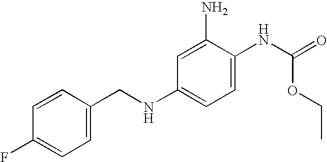

### N-(2-amino-4-(fluorobenzylamino)-phenyl) Carbamic Acid Ethyl Ester

The structure and synthesis of 2-amino-4-(-fluorobenzylamino)-1 ethoxycarbonylaminobenzene is described, for example, in U.S. Pat. Nos. 5,384,330, 5,914,425 and 6,538,151 as well as in Blackburn-Munro et al., CNS Drug Reviews, 11:1-20 (2005), and references cited therein. The terms “2-amino-4-(-fluorobenzylamino)-1-ethoxycarbonylaminobenzene,” “N-(2-amino-4-(4-fluorobenzylamino)-phenyl) carbamic acid ethyl ester” or “retigabine” should be understood to include any pharmaceutically acceptable form of the compound.

Pharmaceutically acceptable forms of an active ingredient include, for example, variations of the referenced active pharmaceutical ingredient that are physiologically tolerable at doses to be administered and retain pharmaceutical activity. Pharmaceutically acceptable forms of an active pharmaceutical ingredient include, for example, solvates, hydrates, isomorphs, polymorphs, pseudomorphs, neutral forms, acid addition salt forms, base salts, esters and prodrugs.

For example, the term “pharmaceutically acceptable acid salts” refers to acid addition salts formed from acids which provide non-toxic anions. The pharmaceutically acceptable anions include, but are not limited to, acetate, aspartate, benzoate, bicarbonate, carbonate, bisulfate, sulfate, chloride, bromide, benzene sulfonate, methyl sulfonate, phosphate, acid phosphate, lactate, maleate, malate, malonate, fumarate, lactate, tartrate, borate, camsylate, citrate, edisylate, esylate, formate, fumarate, gluceptate, glucuronate, gluconate oxalate, palmitate, pamoate, saccharate, stearate, succinate, tartrate, tosylate and trifluoroacetate salts, among a great many other examples. Hemi-salts, including but not limited to hemi-sulfate salts, are likewise directed to the invention. For a review on suitable salts, see “Handbook of Pharmaceutical Salts: Properties, Selection, and Use” by Stahl and Wermuth (Wiley-VCH, Weinheim, Germany, 2002). The pharmaceutically acceptable acid addition salts of the compound of retigabine are prepared using methods well known in the art by treating a solution or suspension of the free base with about one chemical equivalent of a pharmaceutically acceptable acid. Conventional concentration and recrystallization techniques are employed in isolating the salts.

The term “pharmaceutically acceptable solvate” refers to a molecular complex including an active pharmaceutical ingredient and a stoichiometric or non-stoichoimetric amount of one or more pharmaceutically acceptable solvent molecules, including but not limited to water and ethanol. Thus, the term solvate includes a hydrate as one example and an ethanolate as another example.

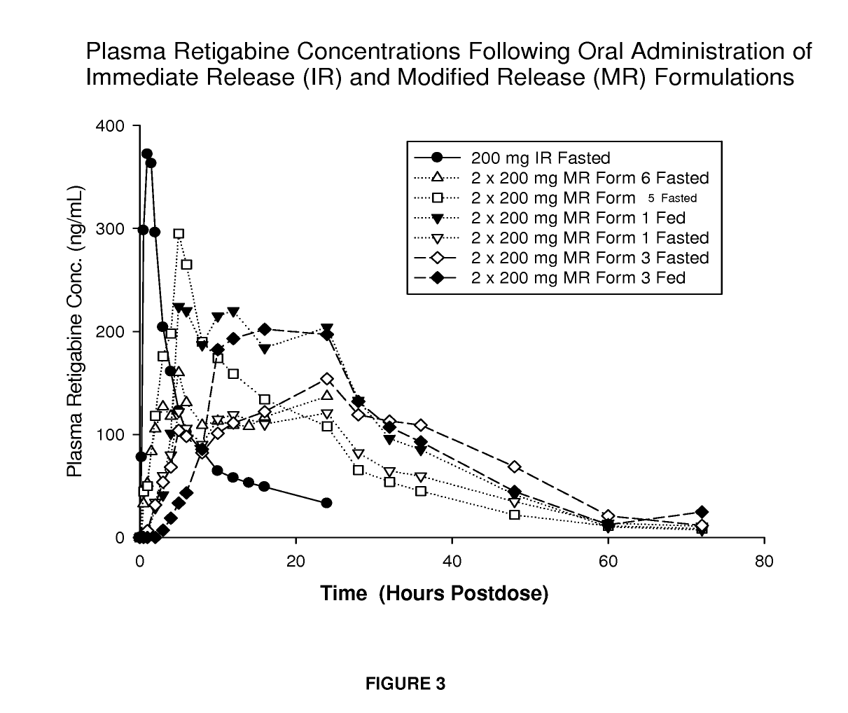

As used herein, the term “sustained” when used in reference to a plasma concentration of an active pharmaceutical ingredient is intended to mean the maintenance of a plasma API concentration within about 50% of the peak plasma concentration for an extended period of time. A sustained concentration includes maintenance of the plasma API concentration within about 48%, 45%, 43%, 40%, 35%, 33%, 30%, 28%, 25%, 23%, 20%, 18%, 15% 12%, 10%, 9%, 8%, 7%, 6%, 5%, 4%, 3%, 2% or 1% of the peak plasma concentration. The term is intended to include minor concentration variations within the prolonged period. A prolonged period of time refers to at least about 3 hours (hrs) and can include periods of 30 hours or more. Exemplary prolonged periods for sustained API plasma concentrations include, for example, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 25 hrs, 26 hrs, 27 hrs, 28 hrs, 29 hrs and 30 hrs or more as well as all periods of time in between these exemplary points. Additionally, a prolonged period of time also can be less than 3 hours so long as there is a recognizable plateau in the API plasma concentration. An example of a sustained concentration is the maintenance of retigabine plasma concentration at about 200 ng/ml beginning from about 8 hours post dose to approximately 30 hours post dose as shown in FIG. 3 (formula 3, fed). FIG. 3 also exemplifies 3 additional sustained concentrations using the pharmaceutical formulations of the invention.

As used herein, the term “drug delivery matrix” is intended to mean an inert substance that provides structural stability and controls the release of an active pharmaceutical ingredient. Drug delivery matrices used in the formulation of the invention include those characterized by a long-lasting, slow and relatively regular incremental release of the active pharmaceutical ingredient upon administration. Examples of drug delivery matrices include non-sucrose fatty acid esters, methylcellulose, ethylcellulose, hydroxypropylmethylcellulose, or polycarbophil.

As used herein, the term “excipient” is intended to mean a pharmaceutically inactive substance. Excipients can be included in a formulation of the invention for a wide variety of purposes and include, for example, pharmaceutically acceptable bulking agents, binders, disintegrants, lubricants, surfactants, drug delivery matrices, release modifying agents, glidants, diluents, vehicles, buffers, stabilizers, tonicity agents, sweeteners, cryoprotectant, lyoprotectant, anti-oxidant, chelating agent and/or preservative. Excipients are well known in the art and can be found in, for example, Remington: The Science and Practice of Pharmacy, (formerly called Remington's Pharmaceutical Sciences), Alfonso R. Gennaro, ed., Lippincott Williams & Wilkins; 20th edition (Dec. 15, 2000).

As used herein, the term “disintegrant” is intended to mean an excipient or a mixture of excipients which promote breakup or disintegration of a solid pharmaceutical formulation such as a tablet or capsule after administration. Therefore, disintegrants are excipients that promote release of a formulation's components, including the active pharmaceutical ingredient. Disintegrants useful in the pharmaceutical formulations of the invention include, for example, a variety of cross-linked cellulose compositions such as crospovidone, croscarmellose sodium and sodium starch glycolate. Other disintegrants well known in the art also can be used in the formulations of the invention and include, for example, corn and potato starch.

As used herein, the term “surfactant” is intended to mean a substance that functions to reduce the surface tension of a liquid in which it is dissolved. Surfactants include, for example, amphiphatic organic compounds that exhibit partial solubility in both organic solvents and aqueous solutions. General characteristics of surfactants include their ability to reduce the surface tension of water, reduce the interfacial tension between oil and also form micelles. Surfactants of the invention include non-ionic and ionic surfactants. Surfactants are well known in the art and can be found described in, for example, Holmberg et al., Surfactants and Polymers in Aqueous Solution, 2d Ed., John Wiley & Sons Ltd. (2003); Surfactants: A Practical Handbook, K. Robert Lange, ed., Hanser Gardner Publications (1999); Vogel, A. I., Vogel's Textbook of Practical Organic Chemistry, 5th Ed., Prentice Hall (1996).

Briefly, non-ionic surfactants include, for example, alkyl poly (ethylene oxide), alkyl polyglucosides such as octyl glucoside and decyl maltoside, fatty alcohols such as cetyl alcohol and oleyl alcohol, cocamide MEA, cocamide DEA, and cocamide TEA. Specific examples of non-ionic surfactants include the polysorbates including, for example, polysorbate 20, polysorbate 28, polysorbate 40, polysorbate 60, polysorbate 65, polysorbate 80, polysorbate 81, polysorbate 85 and the like; the poloxamers including, for example, poloxamer 188, also known as poloxalkol or poly(ethylene oxide)-poly(propylene oxide), poloxamer 407 or polyethylene-polypropylene glycol, and the like, and sucrose esters including, for example, linear or branched, saturated or unsaturated, optionally mono- or polyhydroxylated fatty acids. Polysorbate 20 is synonymous with Tween 20, PEG(20) sorbitan monolaurate and polyoxyethylene (20) sorbitan monolaurate.

Ionic surfactants include, for example, anionic, cationic and zwitterionic surfactants. Anionic surfactants include, for example, sulfonate-based or carboxylate-based surfactants such as soaps, fatty acid salts, sodium dodecyl sulfate (SDS), ammonium lauryl sulfate and other alkyl sulfate salts. Cationic surfactants include, for example, quaternary ammonium-based surfactants such as cetyl trimethylammonium bromide (CTAB), other alkyltrimethylammonium salts, cetyl pyridinium chloride, polyethoxylated tallow amine (POEA) and benzalkonium chloride. Zwitterionic or amphoteric surfactants include, for example, dodecyl betaine, dodecyl dimethylamine oxide, cocamidopropyl betaine and coco ampho glycinate.

As used herein, the term “binder” is intended to mean an excipient or mixture of excipients that impart cohesive qualities, uniform consistency and/or solidification to a solid particle or powdered material, ensuring that a pharmaceutical formulation remains intact after compression and promoting its free-flowing qualities. Binders are also intended to include polymers that modify the release properties and are therefore also include “modified release polymers.” Binders are well known in the art and include, for example, povidone, copovidone, methylcellulose, Hypromellose 2910, polyethylene glycol (PEG) such as PEG 6000 and/or PEG 8000, and hydroxypropylcellulose. Other well known binders applicable to the formulations of the invention include starch, gelatin, and sugars such as sucrose, glucose, dextrose, molasses and lactose, gums such as acacia, sodium alginate, panwar gum, ghatti gum and carboxymethylcellulose.

As used herein, the term “lubricant” is intended to mean an excipient or mixture of excipients that reduce or prevent adhesion of the formulation components to the manufacturing equipment. Lubricants also can reduce interparticle friction, improve rate of flow of the powder substances through manufacturing equipment. An exemplary lubricant useful in the formulations of the invention includes, for example, magnesium stearate. Other lubricants well known in the art also can be used in the formulations of the invention and include, for example, talc, calcium stearate, stearic acid, hydrogenated vegetable oils, sodium dodecyl sulfate and polyethylene glycol (PEG).

As used herein, the term “glidant” is intended to mean a substance which improves the flow characteristics of powder substance. An exemplary glidant which can be used in the formulations of the invention includes, for example, colloidal silicon dioxide.

As used herein, the term “nervous system hyperexcitability” when used in reference to a disorder is intended to mean a state of unusual or excessive nervous system activity. The activity generally is associated with the central nervous system (CNS), but the meaning of the term also includes hyperexcitability of the peripheral nervous system (PNS). Nervous system hyperexcitability also can be characterized by aberrant potassium channel activity including, for example, voltage-gated potassium channels such as KCNQ2, KCNQ3 and/or KCNQ5 potassium channel in mammals. Exemplary disorders characterized by nervous system hyperexcitability include, for example, seizures, epilepsy, convulsions, neuropathic pain, neuralgia, acute and/or chronic reduced cerebral blood supply, neurodegenerative disorders, medicament withdrawal, intoxication and overactive bladder, as well as other disorders exemplified previously. A specific example of a seizure disorder is epilepsy. Specific examples of neuropathic pain include allodynia and hyperalgesa. Specific examples of neuralgia include trigeminal neuralgia (TN), atypical trigeminal neuralgia (ATN), and post-therapeutic neuralgia. Reduced blood supply include, for example, conditions such as stroke and exemplary neurodegenerative disorders include Alzheimer's disease, amyotrophic lateral sclerosis and Parkinson's disease. Overactive bladder includes loss of bladder control such as urinary incontinence, bladder instability, nocturia, bladder hyperreflexia and enuresis.

As used herein, the term “treating,” “treat,” or grammatical equivalents thereof, when used in reference to a disorder or disease is intended to mean preventing, ameliorating or reducing the severity of a clinical symptom indicative of the referenced disorder or disease. Therefore, the term is intended to include administration to inhibit, arrest or mitigate a targeted disorder or symptom as well as prophylactic treatment to forestall development of a targeted disorder or symptom. A specific example of treating a disorder is administration of 2-amino-4-(-fluorobenzylamino)-1-ethoxycarbonylaminobenzene in a formulation of the invention to reduce the severity or frequency of occurrence of a seizure.

As used herein, the term “effective amount” when used in reference to a pharmaceutical formulation of the invention is intended to mean an amount of the active pharmaceutical ingredient to ameliorate at least one symptom associated with a targeted disorder or disease.

As used herein, the term “modified release” means a composition which has been designed to produce a desired pharmacokinetic profile by choice of formulation. For example, the term “modified release” can include delayed, pulsed and sustained release either alone or in any combination.

As used herein, the term “pharmaceutically acceptable” embraces compounds, compositions and ingredients for both human and veterinary use. For example the term “pharmaceutically acceptable salt” embraces a veterinarily acceptable salt. Suitable pharmaceutically acceptable solvates include hydrates.

As used herein, the term “Cₘₐₓ” refers to the mean maximum plasma level concentration.

As used herein the term “AUC” refers to the mean area under the plasma concentration versus time curve over the dosing interval at steady state.

In some embodiments, the present invention provides a modified release pharmaceutical formulation that includes about 30-70% N-(2-amino-4-(fluorobenzylamino)-phenyl) carbamic acid ethyl ester (retigabine), or a pharmaceutically acceptable salt, solvate or hydrate thereof, about 5-30% of a drug delivery matrix that includes hydroxypropylmethylcellulose (HPMC), and an enteric polymer. Formulations of the invention produce a sustained plasma concentration of retigabine following administration to a subject for 4-20 hours longer than the time required for in vitro release of 80% of retigabine.

In some embodiments, the present invention provides a modified release pharmaceutical formulation that includes about 30-70% N-(2-amino-4-(fluorobenzylamino)-phenyl) carbamic acid ethyl ester (retigabine), or a pharmaceutically acceptable salt, solvate or hydrate thereof, about 5-30% of a drug delivery matrix that includes hydroxypropylmethylcellulose (HPMC), about 1.0-10% of an anionic surfactant and an enteric polymer. Formulations of the invention produce a sustained plasma concentration of retigabine following administration to a subject for 4-20 hours longer than the time required for in vitro release of 80% of retigabine.

In some aspects, the invention is directed to a modified release pharmaceutical formulation suitable for use with an active pharmaceutical ingredient. In one embodiment, the modified release formulations are useful for delivering a sustained plasma concentration of retigabine. Retigabine or a pharmaceutically acceptable salt, solvate or hydrate thereof can be formulated in a modified release pharmaceutical formulation of the invention in a wide variety of doses and amounts depending on the intended use and treatment regime. Generally, retigabine can be included in a formulation at between about 30-70% of the total weight of the formulation. More particularly, retigabine or a pharmaceutically acceptable form thereof, can be included in a formulation of the invention at percentages between about 40-60% and between about 49-58%. Retigabine, or a pharmaceutically acceptable form thereof, also can be included at, for example, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68 or 69%, including all values in between these exemplary percentages. The amount of retigabine in a formulation of the invention can therefore include all weights corresponding to these percentages. Exemplary retigabine percentages are described below in the Examples. Retigabine can be administered in a doses ranging from about 5 mg to about 800 mg, including in a range from about 100 mg to about 700 mg, and including in a range from between about 400 mg to about 700 mg. The dose of retigabine can represent quantities used for dosing once daily, twice daily, thrice daily, or more. The doses can include all quantities of retigabine between 5 mg and 800 mg, including, for example, 5 mg, 10 mg, 20 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg and all values in between.

In some embodiments, retigabine can be provided in any of its known polymorphic forms. For example, U.S. Pat. No. 6,538,151, which is incorporated by reference herein in its entirety, describes three retigabine polymorphs, A, B, and C. In some embodiments, formulations of the present invention can utilize pure single polymorphs. For example, polymorph A, in pure form, can be included in formulations of the present invention Likewise, formulations of the present invention can include pure polymorph B or pure polymorph C. In still further embodiments, formulations of the present invention can provide any combination of two or more polymorph forms, such as A and B, or A and C, or B and C, or A, B, and C. Moreover, when combinations of polymorphs are present in formulations of the invention, the polymorphs can be present in any ratio.

A modified release pharmaceutical formulation of the invention also includes a drug delivery matrix. The amount of drug delivery matrix included in a formulation of the invention can assist to prolong retigabine bioavailability for about 4-20 hours or more longer than about 80% of its release at neutral pH. Generally, a drug delivery matrix is included in a formulation of the invention between about 7.5-30% of the total formulation weight. Such a proportion will yield a sustained retigabine plasma concentration following administration to a subject much longer than its release under simulated intestinal conditions. Drug delivery matrices also can be included in a formulation of the invention at percentages between about 10-20% including, for example, about 8, 9, 10, 11, 12, 13, 14, 15, 16, 17, 18, 19, 20, 21, 22, 23, 24, 25, 26, 27, 28 or 29%, as well as all values in between these exemplary percentages. The actual amount of a drug delivery matrix in a formulation of the invention can therefore include all weights corresponding to these percentages. Exemplary percentages of drug delivery matrix are provided below in the Examples.

A specific example of a drug delivery matrix useful in the pharmaceutical formulations of the invention is hydroxypropylmethylcellulose (HPMC). Exemplary types of hydroxypropylmethylcellulose drug delivery matrices include, for example, hypromellose 2208, including Methocel™ K100LV and Methoce™ K100LV CR. Other drug delivery matrices useful in the formulations of the invention include, for example Methocel™ E Premium, Methocel™ K15M Premium, Methocel™ K100LV Premium and ethylcellulose. Other drug delivery matrices include for example, high molecular weight hypromellose (HPMC) 2910 (also known as E), methylcellulose, polyethylene oxide, hydroxypropyl cellulose, xanthan gum, guar gum, Eudragit N M, Eudragit N E, Kollidon S R, galactomannans, dextran, ethylcellulose, carbomer, carbopol, polycarbophil, sodium carboxymethylcellulose, hydroxyethylcellulose, hydroxyethylmethyl-cellulose, shellac, zein, cellulose acetate or combinations thereof. Such drug delivery matrices can be used alone or in combination. Dicalcium phosphate also can be included with the drug delivery matrix.

In some embodiments, the ratio of retigabine to the drug delivery matrix can be in a range from between about 20:1 to about 2:1, including any ratio in between. In some embodiments, the ratio of retigabine to the drug delivery matrix can be in a range from between about 4:1 to about 2:1, including any ratio in between.

The surfactant in a modified release formulation of the invention can be used in proportions up to about 10% of the total composition. Accordingly, surfactants can constitute between about 1.0 to about 10% of the formulation and generally will constitute between about 3 to about 6%, about 3.5 to about 5.5% or about 4 to about 4.5% of the formulation. Surfactants also can be included at, for example, 0.5, 0.75, 1.0, 1.25, 1.5, 1.75, 2.0, 2.25, 2.5, 2.75, 3.0, 3.25, 3.5, 3.75, 4.0, 4.25, 4.5, 4.75, 5.0, 5.25, 5.5, 5.75, 6.0, 6.25, 6.5, 6.75, 7.0, 7.25, 7.5, 7.75, 8.0, 8.25, 8.5 or 8.75%, including all values in between these exemplary percentages. The amount of a surfactant in a formulation of the invention can therefore include all weights corresponding to these percentages. Exemplary percentages of a surfactant are shown herein below and in the Examples for formulations having different total weights. Exemplary surfactants of the invention include the anionic surfactant sodium dodecyl sulfate (SDS) and the non-ionic sucroesters. For example, surfactants in a formulation of the invention can include between about 2-6% sucroester surfactant. In some embodiments, sucroester surfactants can be absent. In further embodiments, a combination of surfactants can be used. Such combinations can include sucroester surfactants or not. Likewise, surfactants in a formulation of the invention can include between about 2-6% SDS surfactant. In some embodiments, SDS surfactant can be absent. In the case of formulations having a combination of surfactants, SDS can be included or not. Following the teachings and guidance provided herein, other surfactants such as those described previously or others well known in the art also can be included in a pharmaceutical formulation of the invention. For example, the anionic surfactant sodium lauryl sulfate can be used in place of SDS.

Disintegrants can be included to constitute up to about 5% of the total formulation, including percentages up to about 4%, 3%, 2% or 1%. Single or multiple disintegrants including two or three or more disintegrants, can be included in a formulation to constitute up to about 10% of the total formulation. For example, one or more disintegrants can be included in a formulation at a percentage between about 0.5-5.5%, 1-5.0%, 2-4.5%, 2.5-4.0% or 3.0-3.5% as well as all ranges in between these values up to about 5% each of the total formulation. Exemplary disintegrants applicable in a formulation of the invention include, for example, crospovidone, croscarmellose sodium or a combination thereof. Accordingly, a pharmaceutical formulation of the invention can include, for example, 0.5, 1.0, 1.5, 2.0, 2.5, 3.0, 3.5, 4.0, 4.5 or 5.0% crospovidone as well as all values in between these percentages. A pharmaceutical formulation of the invention also can include, for example, 0.5, 1.0, 1.5, 2.0, 2.5, 3.0, 3.5, 4.0, 4.5 or 5.0% croscarmellose sodium as well as all vales in between these percentages. These exemplary disintegrants as well as others known in the art can be included individually or in any combination thereof up to about 10% of the total formulation. Specific examples of disintegrant amounts and combinations of a formulation of the invention include 0.5-5.5% crospovidone, croscarmellose sodium or a combination thereof comprises 0.5-2.5% crospovidone, 2.0-5.5% croscarmellose sodium or 0.5-2.5% crospovidone and 2.0-5.5% croscarmellose sodium.

A modified release pharmaceutical formulation of the invention can further include a wide variety of excipients. Excipients are well known in the art and are useful to facilitate, for example, manufacturing processes, dosage amounts and delivery of the active pharmaceutical ingredient. Exemplary excipients of the formulations of the invention have been described above and further below in Table 1. Such excipients include, for example, binders, disintegrants, surfactants, lubricants and glidants.

A further excipient that can be included in a formulation of the invention includes binders. One or more binders can be included in a formulation of the invention to constitute up to about 40% of the total formulation weight including percentages up to about 35%, 30%, 25%, 20%, 15%, 10% or 5%. A single binder can be included in a formulation, or alternatively, two, three, or four or more different binders can be included to constitute the total percentage of binders in the formulation. For example, one or more binders can be included in a formulation of the invention at a percentage between about 5-40%, 20-35%, 25-30% as well as within ranges between about 1-6%, 1-5%, 1-4%, 2-5% or 3-5% including all ranges in between and above these values up to about 40% of the total formulation by weight. Exemplary binders applicable in the formulations of the invention include for example, microcrystalline cellulose, hypromellose 2910, copovidone, povidone, starch and polyethlylene glycol as well as all combinations thereof up to about 40% of the total formulation by weight. Exemplary amounts of binders and combinations thereof applicable in the formulations of the invention include, for example, about 5-40% microcrystalline cellulose, 0-10% hypromellose 2910, 0-10% copovidone, 0-10%, polyethlylene glycol.

Therefore, a pharmaceutical formulation of the invention can include, for example, 1, 3, 5, 10, 15, 20, 25, 30, 35 or 40% microcrystalline cellulose as well as all values in between these percentages. A formulation of the invention also can include, for example, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 or 10% hypromellose 2910 as well as all values in between these percentages. Additionally, a formulation of the invention also can include, for example, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 or 10% copovidone as well as all values in between these percentages. Binders such as polyethylene glycol and the like can additionally be included in a formulation of the invention at, for example, 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5 or 10%, including all values in between these percentages. These exemplary binders as well as others known in the art can be included individually or in any combination thereof up to about 40% of the total formulation. Specific examples of binder amounts and combinations of a formulation of the invention are 25-30% microcrystalline cellulose, 25-30% microcrystalline cellulose and 3-5% copovidone, 25-30% microcrystalline cellulose and 1-4% hypromellose 2910 or 25-30% microcrystalline cellulose, 1-4% hypromellose and 3-5% copovidone. A number of other specific examples of binder amounts and combinations thereof are exemplified further below in Tables 1-3.

Lubricants and glidants also can be included in a modified release pharmaceutical formulation of the invention to constitute up to about 2% or more for each excipient. Accordingly, percentages of up to about 0.25, 0.5, 0.75, 1.0, 1.25, 1.5, 1.75 or 2.0% for a lubricant or for glidant can be included in a formulation. Various combinations of two or three or more different lubricants or two or three or more glidants also can be included in a formulation of the invention up to about 2% for each excipient. An exemplary lubricant useful in the formulations of the invention includes, for example, magnesium sterate. An exemplary glidant useful in the formulations of the invention includes silicone dioxide such as colloidal silicone dioxide. Specific examples of lubricant and glidant amounts in a formulation of the invention include 0.5-2.0% magnesium stearate and 0.25-1.5% silicone dioxide, respectively.

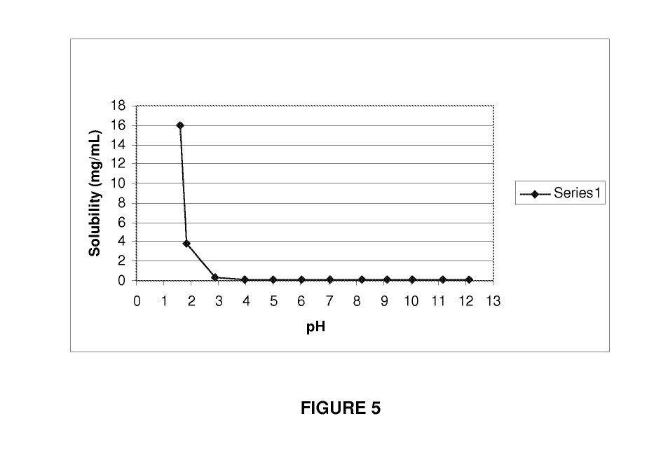

In some embodiments, a formulation of the invention includes about 30-70% N-(2-amino-4-(fluorobenzylamino)-phenyl) carbamic acid ethyl ester (retigabine), or a pharmaceutically acceptable salt, solvate or hydrate thereof in about 5-30% of a drug delivery matrix. The formulations also include an agent for retarding release in the gastric environment. The resultant formulation exhibits a plasma concentration vs. time profile that is substantially flat over an extended period lasting for about 4 to about 36 hours, as shown for example, in FIG. 3 and in Tables 5 and 6 below. The agent for retarding release in the gastric environment can also delay the solubility of retigabine. As seen in FIG. 5, the solubility of retigabine drops off precipitously above pH 3. By bypassing the gastric environment, for example, by use of an enteric polymer, retigabine is first exposed to an environment of the lower intestine which is at a higher pH than the stomach. Furthermore, the pH in the lower intestine is typically in a range higher than where retigabine exhibits good solubility.

In some embodiments, the agent that retards the release into the gastric environment includes an enteric polymer. Most enteric polymers operate by presenting a surface that is stable at the pH found in the stomach. However, such polymers tend to break down at less acidic pH, such as that found in the lower intestine. Materials that can be used as enteric polymers include fatty acids, waxes, and shellac as well as plastics. In some embodiments, the enteric polymer is selected from polyvinylacetate phthalate, hydroxypropylmethylcellulose acetate succinate (HPMC-AS), and a copolymer of two or more of methyl methacrylate, methacrylic acid, ethyl acrylate, and methyl acrylate. In some embodiments, the enteric polymer is selected from cellulose acetate phthalate, cellulose acetate succinate, methylcellulose phthalate, ethylhydroxycellulose phthalate, polyvinylacetatephthalate, polyvinylbutyrate acetate, vinyl acetate-maleic anhydride copolymer, styrene-maleic mono-ester copolymer, methyl acrylate-methacrylic acid copolymer, and methacrylate-methacrylic acid-octyl acrylate copolymer. Any of the foregoing enteric polymers can be used either alone or in combination, or together with other polymers that can serve as agents to retard the release into the gastric environment.

The enteric polymer can be used in conjunction with other substances to modify the release properties of the formulation, such as alkyl cellulose derivatives as exemplified by ethyl cellulose, crosslinked polymers such as styrene-divinylbenzene copolymer, polysaccharides such as dextran, cellulose derivatives which are treated with bifunctional crosslinking agents such as epichlorohydrin, dichlorohydrin, 1,2-, 3,4-diepoxybutane, etc. The enteric polymer can also be used in conjunction with starch and/or dextrin. The agent retarding release in the gastric environment can further include a delivery matrix as described herein above or selected from hydroxypropylmethylcellulose, hydroxypropylcellulose, polyethylene oxide, and a copolymer of polyvinylacetate and polyvinylpyrrolidone.

In some embodiments, the enteric polymer materials are pharmaceutically acceptable methacrylic acid copolymers and the like possessing anionic character. Exemplary copolymers are based on methacrylic acid and methyl methacrylate, for example having a ratio of free carboxyl groups; methyl-esterified carboxyl groups of 1:>3, e.g. around 1:1 or 1:2, and with a mean molecular weight of 135,000. Such polymers are sold under the trade name Eudragit™, such as the Eudragit L series e.g. Eudragit L 12.5™, Eudragit L 12.5P™, Eudragit L100™, Eudragit L 100-55™, Eudragit L-30™, Eudragit L-30 D-55™, the Eudragit S™series e.g. Eudragit S 12.5, Eudragit S 12.5™, Eudragit S100™, the Eudragit NE™ series e.g. Eudragit NE 30D™, the Eudragit RL™ series, e.g. Eudragit RL 12.5™, Eudragit RL 100™, Eudragit RL PO™, Eudragit RL 30D™, and the Eudragit RS™ series e.g. Eudragit RS 12.5™, Eudragit RS 100™, Eudragit RS PO™, and Eudragit RS 30D™. Convenient aqueous application of these enteric polymers can be accomplished using Acryl-Eze® (Colorcon, Inc.; West Point, Pa.).

The aforementioned enteric polymers can be used alone or in conjunction with a plasticizer. Aqueous plasticizers that can be used include propylene glycol or Citroflex™ or Citroflex A2™ which is mainly triethyl citrate or acetyl triethyl citrate. Non-aqueous plasticizers also include the above mentioned aqueous plasticizers as well as diethyl and dibutyl phthalate and dibutyl sebacate. The enteric polymer can also be used in conjunction with an anti-tack agent such as talc, silica or glyceryl monostearate. The enteric polymer can be used in conjunction with between about 10 to about 25 wt. % plasticizer based on the total coating weight and up to about 50 wt % of an anti tack agent, including, for example, between about 5 to about 20 wt. % of anti-tack agent based on the total coating weight.

The invention further provides a pharmaceutical formulation that includes 30-70% N-(2-amino-4-(fluorobenzylamino)-phenyl) carbamic acid ethyl ester (retigabine), or a pharmaceutically acceptable salt, solvate or hydrate thereof, 7.5-30% drug delivery matrix, 0.5-10% disintegrant, an excipient and an enteric coating, the pharmaceutical formulation producing a sustained plasma concentration of the retigabine for about 4-20 hours longer than the time required for in vitro release of 80% of the retigabine following administration to a subject.

Given the teachings and guidance provided herein excipients other than those exemplified above and known in the art also can be included in a modified release pharmaceutical formulation of the invention. There are a wide variety of excipients having various useful functions in, for example, the manufacture, storage and/or delivery of a pharmaceutical formulation. Any of such excipients can be included in a formulation of the invention so long as its addition or substitution does not substantially alter the ability of the formulations of the invention to produce a sustained plasma concentration of active pharmaceutical ingredient for about 4-20 hours longer than the time required for in vitro release of the active ingredient (retigabine) under simulated in vivo conditions. In addition, excipients such as pharmaceutically acceptable carriers, including auxiliary substances, carriers and/or diluents also can be included in a formulation of the invention. Examples of such other excipients include dicalcium phosphate, and enteric coatings such as Eudragit™ or Acryl-Eze® (available through Evonik Industries and Colorcon). Pharmaceutical formulations of the invention containing various combinations and proportions of some or all of the above components are exemplified further below in the Examples and in Tables 1-3.

Pharmaceutical formulations of the invention having the components exemplified herein result in a modified release of the active pharmaceutical ingredient such that a plateau or an approximate peak plasma concentration is sustained for an extended period to time compared to immediate release or compared to slow release formulations. FIG. 3 illustrates such sustained plasma concentrations for a few exemplary formulations of the invention in both the fed and fasted state. As shown therein, active ingredient rises to an approximate maximum concentration within about 2-5 hrs or more depending on the specific formulation and whether the individual is in a fed or fasted state. Concentrations approaching an approximate maximum concentration are sustained out to about 25-30 hrs.

Accordingly, the modified release pharmaceutical formulations of the invention can deliver a sustained plasma concentration from about 3 to about 36 hrs, from about 3 to about 28 hrs, from about 4 to about 25 hrs, from 5 to about 20 hrs, from 6 to about 15 hrs or about 5 to about 10 hrs. In general, formulations of the invention can produce a sustained plasma concentration of retigabine following administration to a subject for 4-20 hours longer than the time required for in vitro release of 80% of retigabine. The observed 4-20 hour sustained plasma concentration in vivo relative to the in vitro dissolution profile under simulated in vivo conditions for modified release formulations of the invention was unexpected.

The in vitro release of retigabine under simulated in vivo conditions involves subjecting the retigabine formulation to a period of exposure to acidities that can simulate gastric conditions. For example, in FIG. 4 and Example V below, gastric conditions are simulated by initial exposure of the retigabine formulation to 0.1 N HCl for one hour. Formulations of the invention that incorporate an enteric polymer are expected to exhibit minimal release of retigabine under these conditions as shown in FIG. 4 and Example V. After the initial exposure to 0.1 N HCl, to simulate exposure to gastric conditions, the sample is then exposed to a pH 7.5 borate buffer system. It will be apparent to one of skill in the art, that these conditions are merely exemplary and other conditions can be employed in a simulation. For example, other buffer systems can employed.

In vitro dissolution profiles can be obtained under conditions that are designed to be indicative of in vivo performance or bioavailability. Such in vitro conditions are well known in the art and include, for example, the borate buffer system employed in Example V. Under such conditions, or other similar conditions well known in the art, a longer in vitro dissolution profile can be expected to be indicative of prolonged in vivo release or bioavailability. However, in accordance with the present invention, there is an observed disconnect between the in vitro dissolution profile observed under conditions indicative of in vivo performance and the observed in vivo profile.

In contrast, one skilled in the art will recognize that in vitro release conditions can also be designed for other purposes. For example, dissolution rates can be altered using conditions modified to achieve a particular goal other than being indicative of in vivo performance, such as to measure solubility or to decrease the time for dissolution. Such modified dissolution conditions can include, for example, increased amounts of surfactant, change paddle speeds, and/or use flow-through techniques to modulate the in vitro dissolution rates. Given the teachings and guidance provided herein as well as that well known in the art, those skilled in the art will understand that dissolution results obtained from such modified conditions may not be useful as an indicator of in vivo bioavailability.

Exemplary sustained plasma concentrations of the active pharmaceutical ingredient produced from single dose modified release formulations of the invention include, for example, at least about 20 ng/ml after administration once a day, at a dosage of about 400 mg, in the fed or fasted state and more particularly at least about 50, 100, 150, 200, 250, 300 or 350 ng/ml or higher, at a dosage of about 400 mg. In particular, exemplary formulations of the invention produce a Cₘₐₓ, in the fasted state, between about 100 ng/mL to about 300 ng/mL, or within a 90% confidence interval thereof. As described further below in the Examples, exemplary area under the concentration of retigabine in plasma versus time curve (AUC) after administration in the fasted or fed state can be used to assess the sustained concentration of active ingredient. For example, for formulations administered once per day at 400 mg, the formulations of the invention provide an AUC_{0-inf} value in the fasted state that is in a range from between about 3000 ng-hr/L to about 7000 ng-hr/L. In other embodiments the AUC_{0-inf} can be between about 4000 ng-hr/L to about 6800 ng-hr/L, and between about 4000 ng-hr/L to about 10,000 ng-hr/L in further embodiments. One skilled in the art will recognize the ability to obtain similar results for Cₘₐₓ and AUC_{0-inf} by altering the frequency in conjunction with altering the quantity of dosages. Similarly, one skilled in the art also will recognize that the observed Cₘₐₓ and/or AUC_{0-inf} values can vary with different dosage amounts and frequency compared to the above exemplary values without substantially affecting the modified release performance of the formulations as they are exemplified herein. Dosages can be formulated for administration every other day, twice-daily, three times daily, and four times daily, for example, without substantially altering Cₘₐₓ and the AUC results shown for the 400 mg dose. In addition to sustained plasma concentrations, the modified release formulations of the invention also exhibit a steady rate of clearance compared to immediate release formulations.

The modified release formulations of the invention release at least a portion of the active pharmaceutical ingredient from between about 0.5 to 2 hours after administration. However, the modified release formulations can also be used in conjunction with an enteric coating that can delay the release of at least a portion of the active pharmaceutical ingredient from between about 4 to 6 hours. This can be beneficial by allowing slower sustained release in the intestine. This can be useful in reducing side effects by effectively lowering Cₘₐₓ, while still assuring prolonged bioavailability of the active ingredient. Release of an active pharmaceutical ingredient refers to the amount or percentage of free compound that is dissociated or relinquished from other components in the formulation, which then subsequently dissolve. In comparison, immediate release formulations result in greater than 90% of the active ingredient within the first hour following administration. In certain embodiments, the modified release formulations release no greater than 90% of the active pharmaceutical ingredient from the formulation during the first 2 hours after administration. In other embodiments, the formulations of the invention release no greater than 80%, no greater than 70% or no greater than about 60% of the active pharmaceutical ingredient during the first 2 hours following administration. For example, the time to release at least about 80% of an active pharmaceutical ingredient can be, for example, at least about 4 hours. The release rates of exemplary formulations of the invention are illustrated in FIGS. 2 and 3. In some embodiments, the release of the active ingredient in vivo is between about 3 to 6 hours after in vitro release.

Methods for assessing the amount or rate at which an active ingredient is released from a formulation are well known in the art. Exemplary methods include, for example, EA residual and direct tests. Briefly, the residual test measures the amount of active ingredient remaining in a formulation following selected time periods in solution. Subtraction of the amount released at each time period from the amount initially present for each time period provides the rate of release. The direct test measures the concentration of active pharmaceutical ingredient in the dissolution medium at each time point to calculate the rate or amount of release. Exemplary release rates of an active pharmaceutical ingredient from the formulations of the invention range from about 8 to 100% at 0.5 hours, 18 to 100% at 1 hour, 34-100% at 2 hours, 53-100% at 3 hours and 66-100% at 4 hours however more detailed release rate information is provided in the Examples below.

The formulations of the invention can be characterized by a plasma concentration versus time profile having a substantially flat portion that lasts between about 4 to about 36 hours in some embodiments, and between about 10 and 20 hours in other embodiments. Without being bound by theory, the extended period of time at which the plasma level of retigabine is at Cₘₐₓ can relate to a biological mechanism such as recirculation. For example, numerous drugs undergo enterohepatic recycling which involves elimination via the bile in an unchanged or conjugated form. Drugs secreted into the bile enter into the gall bladder, which is periodically emptied into the small intestine. Entry into the small intestine provides a means by which the drug is absorbed back into the body and prolongs the time required for the drug to be eliminated from the body.

Again, without being bound by theory, the extended period of time at which the plasma level of retigabine is at Cₘₐₓ can relate to the formation of a quasi-stable complex between retigabine and the delivery matrix. Still another reason for the extended period of time at which the plasma level of retigabine is at Cₘₐₓ can relate to a combination of enterohepatic recirculation and complex formation. Yet another reason for the extended period of time can relate to the solubility profile of retigabine. Under the influence of an enteric polymer, the retigabine formulation bypasses the more acidic environment of the stomach and enters the lower intestine where the acidity is high enough impact drug solubility and systemic release.

The modified release pharmaceutical formulations of the invention can be manufactured into a dry powder pharmaceutical including into a variety of different solid dosage forms well known in the art. Solid dosage forms are particularly useful for delivering an accurate dosage to a specific site, usually orally, but also can be administered sublingually, rectally or intravaginally. Solid dosage forms include, for example, tablets, pills, chewable tablets, capsules, caplets, pellets or granules and the like.

The modified release pharmaceutical formulations of the invention can be manufactured to contain any desired solid dosage amount of an active pharmaceutical ingredient and in any desired total weight of the solid dosage form so long as the component proportions set forth herein are retained in the final dosage form. The active pharmaceutical ingredient can be, N-(2-amino-4-(fluorobenzylamino)-phenyl) carbamic acid ethyl ester or a compound having solubility characteristics similar to N-(2-amino-4-(fluorobenzylamino)-phenyl) carbamic acid ethyl ester. For example, solid dosage forms can be manufactured to contain 5, 10, 15, 25, 50, 75, 100, 125, 150, 175, 200, 225, 250, 275, 300, 325, 350, 375, 400, 450, 500, 550, 600, 650, 700, 750, 800 mg or more of active ingredient per dosage form, including any value in between. Exemplary total weight of a dosage form can include, for example, 25, 50, 100, 150, 200, 250, 300, 350, 400, 450, 500, 550, 600, 650, 700, 750, 800, 850, 900, 1000 mg or more. All weights in between, above and below these exemplary amounts of active ingredient and total weights also can be manufactured given the teachings and guidance provided herein. Because the modified release formulations of the invention result in a sustained plasma concentration they are particularly useful in dosage forms prepared to have an effective amount of active pharmaceutical ingredient for administration three times daily (TID, twice daily (BID), once daily (QD), every other day, three times weekly, twice weekly, once a week or for longer dosing periods. Such lower dosing regimes similarly promote greater patient compliance. Such solid dosage forms can be packaged and stored following pharmaceutical practices known in the art.

Methods for manufacturing dry powder pharmaceuticals well known in the art can be used for the production of a modified release pharmaceutical formulation of the invention. Such methods include, for example, direct compression, mixing and/or granulation. Powder formulations that can be mixed well can be, for example, compressed into a tablet or other solid dosage form by direct compression. Mixing includes, for example, convective mixing, shear mixing and/or diffusive mixing. Granulation methods including, for example, wet granulation, dry granulation, fluidized bed granulation and extrusion granulation, can be used for manufacturing other powder formulations, followed by compression into a table or other solid dosage form.

Formulation homogeneity can be improved by, for example, wet or dry milling to reduce particle size and/or by, for example, combining and blending formulation components in stages. For example, an active pharmaceutical ingredient can be granulated with one or more of the components by, for example, dry or wet granulation, and then blended with the remaining components. Alternatively, an active pharmaceutical ingredient can be, for example, first dry blended with one or more drug delivery matrices, while other excipients, such as glidants, lubricants and the like, are be subsequently admixed in one or more blending operations. If desired, prior to blending one or more of the components can be sized by screening or milling or both. To prepare the final drug product, the compressed dosage forms can undergo further processing, such as coating, polishing, and the like. For a discussion of dry blending, wet and dry granulation, milling, screening, tableting, coating, and the like, as well as a description of other methods known in the art for preparing pharmaceutical compositions, see A. R. Gennaro (ed.), Remington: The Science and Practice of Pharmacy (20th ed., 2000); H. A. Lieberman et al., (ed.), Pharmaceutical Dosage Forms: Tablets, Vol. 1-3(2d ed., 1990); and D. K. Parikh & C. K. Parikh, Handbook of Pharmaceutical Granulation Technology, Vol. 81 (1997).

Formulations manufactured using the above methods are exemplified further below in the Examples. Accordingly, the invention provides a method of preparing a pharmaceutical formulation. In specific exemplary embodiments, the method includes mixing a milled active pharmaceutical ingredient such as N-(2-amino-4-(fluorobenzylamino)-phenyl) carbamic acid ethyl ester with a drug delivery matrix, a surfactant and a binder, for example, and/or other components exemplified herein in proportions exemplified above or set out below in Tables 1-3. The mixing process is followed by compressing the mixture in an appropriate shape tablet. The tablet, capsule or other dosage form may then be optionally completed with an enteric coating or other types of coating. In other specific exemplary embodiments, the method includes wet granulation methods of preparing a pharmaceutical formulation of the invention such as the method exemplified below in Example II. The granulation can be performed in a high share mixer or fluid bed dryer. This exemplary formulation also is lubricated and compressed to prepare a desired dosage form. The dosage form may be optionally completed with an enteric coating. Pharmaceutical formulations prepared by the methods of the invention exhibit long-term stability of the active ingredient suitable for storage or immediate use.

The solid dosage forms of a pharmaceutical formulation of the invention are useful for delivering a controlled amount of active pharmaceutical ingredient over a sustained period of time. Accordingly, the invention provides a method of controlling the release of an active pharmaceutical ingredient. The method includes administering to an individual a pharmaceutical formulation having 30-70% active pharmaceutical ingredient, 1-30% drug delivery matrix, up to 9% surfactant and an excipient, the pharmaceutical formulation producing a sustained plasma concentration of the active pharmaceutical ingredient for about 4-20 hours following administration to an individual, the active pharmaceutical ingredient retigabine or a compound having solubility characteristics substantially similar to that of N-(2-amino-4-(fluorobenzylamino)-phenyl) carbamic acid ethyl ester, or a pharmaceutically acceptable salt, solvate or hydrate thereof.

Also provided is a method of treating a disorder characterized by nervous system hyperexcitability. The method includes administering to a subject in need thereof an effective amount of a pharmaceutical formulation having 30-70% active pharmaceutical ingredient, 1-30% drug delivery matrix, up to 9% surfactant and an excipient, the pharmaceutical formulation producing a sustained plasma concentration of the active pharmaceutical ingredient for about 4-20 hours following administration to the subject, the active pharmaceutical ingredient comprising retigabine or a compound having solubility characteristics substantially similar to that of N-(2-amino-4-(fluorobenzylamino)-phenyl) carbamic acid ethyl ester, or a pharmaceutically acceptable salt, solvate or hydrate thereof.

Active ingredients having a structure of retigabine or compound with similar structure and/or solubility profile can be included in a pharmaceutical formulation of the invention for the treatment of a wide range of disorders characterized by nervous system hyperexcitability. Such disorders include, for example, seizure, seizure disorders such as epilepsy, convulsions, and neuropathic pain as well as those exemplified further below. Compounds including the 1,2,4-triaminobenzene derivatives related to retigabine have been described to treat these and other disorders or diseases characterized by nervous system hyperexcitability. Employing the modified release pharmaceutical formulations in conjunction with retigabine or related compounds is particularly useful because it provides for lower dosing and greater efficacy due to the production of a long lasting sustained plasma concentration.

For example, compounds such as retigabine are effective for treating or reducing the severity of seizures, epileptic seizures, benign familial neonatal convulsions which is an inherited form of epilepsy, complex partial seizures, convulsions and/or other seizure disorders (see, for example, U.S. Pat. No. 5,384,330; Bialer et al., Epilepsy Research 34:1-41 (1999); Blackburn-Munro and Jensen, Eur. J. Pharmacol. 460:109-116 (2003); Wickenden et al., Expert Opin. Ther. Patents 14:1-13 (2004); Porter et al., Neurotherapeutics 4:149-154 (2007); Rogawski, Trends in Neurosciences 23:393-398 (2000)).

Retigabine and related compounds, such as flupirtine, also are effective for treating or reducing the severity of neuropathic pain (see, for example, U.S. Pat. No. 6,117,990, including references cited therein, and Blackburn-Munro and Jensen, supra), including, for example, allodynia, hyperalgesia and phantom limb pain. Allodynia refers to the perception of stimuli which are not painful per se, such as contact or heat/cold, as pain. Hyperalgesic refers to the feeling of painful stimuli more strongly than a normal person. Phantom pain refers to the perception of pain which is non-existent. The terms reflex sympathetic dystrophy (RSD) and sympathetically maintained pain (SMP) are furthermore used. Therefore, retigabine or related compounds included in a modified release pharmaceutical formulation of the invention are useful to treat disorders manifested by lower pain thresholds as well as disorders manifested by higher pain sensations. There are a wide variety of disorders and diseases causing neuropathic pain. Exemplary causes include, for example, viral infection such as Herpes Zoster which produces postherpetic neuralgia (PHN), a painful and common complication of shingles, Acquired Immune Deficiency Syndrome, burn wounds, cancer, cytostatic or cytotoxic treatment of cancer, nerve damage and/or nerve compression.

Promotion of other effects useful for retigabine or related compounds in a modified release formulation of the invention include, for example, those which are useful for the treatment of pain such as muscle relaxation, fever reduction and/or peripheral analgesia (see, for example, U.S. Pat. Nos. 5,384,330; 6,326,385). Retigabine or related compounds in a modified release formulation of the invention are further useful to promote a neuroprotective effective useful for treating, for example, neurodegenerative disorders and/or stroke as well as secondary or aftereffects of acute or chronic reduced cerebral blood supply such as those caused by neurodegenerative disorders and stroke (see, for example, U.S. Pat. No. 5,852,053). Exemplary neurodegenerative disorders applicable for treatment with retigabine or related compounds as the active ingredient in a modified release formulation of the invention include, for example, Alzheimer's disease, Huntington's chorea, multiple sclerosis, amyotrophic lateral sclerosis, Parkinson's disease, infection-related encephalopathy including encephalopathy mediated by an infection from Human Immunodeficiency virus, rubella viruses, herpes viruses and borrelia, Creutzfeld-Jakob disease, trauma-induced neurodegeneration or neuronal hyperexcitation state, withdrawal from intoxication, a disorder of the peripheral nervous system and/or a polyneuropathy or polyneuritide disorder.

Other therapeutic applications useful for a modified release formulation of the invention having an active ingredient of retigabine or related compounds include, for example, conditions caused by aberrant or undesirable smooth muscle contraction. As described above, retigabine or related compounds are useful to inhibit smooth muscle contraction. Conditions exhibiting undesirable smooth muscle contraction include, for example, irritable bowel syndrome, chronic obstructive pulmonary disease (COPD), gall bladder disorders, hypertension and esophageal hyperactivity.

Further, one molecular site of action for retigabine or related compounds, such as flupirtine, includes potassium channels. For example, N-(2-amino-4-(fluorobenzylamino)-phenyl) carbamic acid ethyl ester is a potassium channel modulator which activates or opens voltage-gated potassium channels. Channel opening results in reduced neuronal excitability and/or lower neurotransmitter release for the potassium KCNQ2/3 channel, for example (Delmas and Brown, Nat. Revs Neurosci. 6:850-62 (2005); Wickenden et al., Mol. Pharmacol. 58:591-600 (2000); Main et al., Mol. Pharmcol. 58:253-62 (2000); Wuttke et al., Mol. Pharmacol. 67:1009-17 (2005)). Additionally, compounds such as N-(2-amino-4-(fluorobenzylamino)-phenyl) carbamic acid ethyl ester have been shown to increase neuronal M currents and to increase the channel open probability of KCNQ 2 and or KCNQ 3 channels (collectively “KCNQ⅔” channels; Delmas and Brown, (supra)). Disorders caused or exacerbated by increased neuronal excitability, decreased potassium channel opening and/or decreased neuronal M currents can therefore be treated using a modified release formulation of the invention having a 1,2,4-triaminobenzene derivative of formula I as an active ingredient. Such disorders can be characterized by the activation of voltage-gated potassium channels by a modified release formulation of the invention to alleviate the occurrence or severity of one or more symptoms.

Treatment of any of the above disorders or diseases can be accomplished by administering a modified release formulation of the invention having an effective amount of an active ingredient. Effective amounts include an amount sufficient to alleviate at least one symptom and can vary depending on the disorder and the desired treatment regime. Effective amounts can range from about 5-1,500 mg per day or from about 0.1-5.0 mg/kg per dose. For example, a subject can be administered a modified release formulation of the invention having an effective amount of an active ingredient between about 10-1,200 mg, 20-1,000 mg, about 30-800 mg, about 40-600 mg, about 50-400 mg, about 60-200 mg or about 70-100 mg per day. Other effective amounts of an active ingredient in a modified release formulation of the invention include, for example, 1.0, 2.5, 5.0, 7.5, 10, 12, 15, 18, 20, 22, 25, 28, 30, 32, 35, 38, 40, 45, 50, 55, 60, 65, 70, 75, 80, 85, 90, 95 or 100 mg per day. Other effective amounts of an active ingredient in a modified release formulation of the invention include, for example, 400, 425, 450, 500, 550, 600, 650, 700, and 750 mg per day. All amounts in between the above exemplary effective amounts also can constitute an effective amount of an active ingredient in a modified release formulation of the invention. Similarly, those skilled in the art will understand that the corresponding amount per weight to those amounts exemplified above also can be used as a measure of an effective amount.

An effective amount will generally be delivered in dosing periods of about three times daily (TID, twice daily (BID), once per day (QD), thrice weekly, twice weekly or in greater dosing intervals. However, depending on the dosage form an effective amount also can be delivered in more frequent dosing intervals including, for example, two or more times a day or 4, 5 or 6 times a week.

Similarly, the modified release pharmaceutical formulations of the invention are also applicable to a variety of different modes of administration. The modified release formulations are exemplified herein as solid dosage forms to be, for example, orally administered. However, those skilled in the art will understand that such solid dosage forms also can be admixed with a pharmaceutical carrier, liquid diluent or syrup, for example, administered by other routes. Dilution into a liquid pharmaceutically acceptable medium can occur immediately prior to administration or prior to substantial release of the active ingredient. Particularly useful media include, for example, a buffer or other solution having a pH that retards or inhibits release of the active ingredient. Given the teachings and guidance provided herein, those skilled in the art will understand that a variety of different dosing intervals and even modes of administration are applicable for use with a modified release formulation of the invention.

Therefore, the invention further provides a method of treating disorder characterized by nervous system hyperexcitability wherein the disorder includes a seizure disorder, neuropathic pain, a neurodegenerative condition or a disorder characterized by activation of voltage-gated potassium channels or aberrant smooth muscle contraction. The modified release formulations of the invention also can be used to produce, for example, an anti-seizure, muscle relaxing, fever reducing, peripherally analgesic or anti-convulsive effect. Other effects include increase the channel opening probability of KCNQ2/3 channels or increasing neuronal M currents.

In some embodiments, the present invention provides an oral dosage form that includes retigabine or a pharmaceutically acceptable salt or solvate thereof, which provides a mean (over a patient group) flattened plasma profile for an extended period of time, for example about 4 to about 24 hours, for example about 4 to about 15 hours, and in some embodiments, about 4 to about 12 after administration. Such a dosage form is considered to be suitable for twice daily or even once daily administration. Currently retigabine immediate release (IR) tablets are administered three times a day. Such a dosage form is also indicated for administration in both fasted and fed states, with substantially no clinically relevant food effect, i.e. no dose dumping under fed conditions. Additionally the oral dosage forms of the present invention can provide less interpatient variability in the pharmacokinetics than previous modified release formulations of retigabine.

In some embodiments, the present invention provides an oral dosage form that includes: (i) an erodable core, which core comprises a first modified release composition including retigabine or a pharmaceutically acceptable salt or solvate thereof, and a pharmaceutically acceptable carrier therefore; and (ii) an erodable coating around said core, which coating includes one or more openings extending substantially completely through said coating but not penetrating said core and communicating from the environment of use to said core, wherein release of retigabine or a pharmaceutically acceptable salt or solvate thereof, from the erodable core occurs substantially through the said opening(s) and through erosion of said erodable coating under pre-determined pH conditions.

In further embodiments, the invention provides an oral dosage form wherein (i) the erodable core includes a first modified release composition and a second composition such that each composition includes retigabine or a pharmaceutically acceptable salt or solvate thereof, and a pharmaceutically acceptable carrier therefore, such that the first and second compositions are arranged to release drug at differing release rates on administration such that the rate of release of the drug from the dosage form is substantially independent of pH and (ii) an erodable coating around the core, which coating includes one or more openings extending substantially completely through the coating but not penetrating the core and communicating from the environment of use to the core, wherein release of retigabine or a pharmaceutically acceptable salt or solvate thereof, from the erodable core occurs substantially through the opening(s) and through erosion of the erodable coating under pre-determined pH conditions.

In one aspect, a modified release composition provides delayed release of retigabine or a pharmaceutically acceptable salt or solvate thereof. The release rate of the drug from the second composition is substantially greater than from the first composition. In some embodiments, the second composition is an immediate release composition. The erodible coating can be an enteric coating layer, such as a non-permeable enteric coating layer.

The second composition can be formulated so that it provides immediate release of retigabine or a pharmaceutically acceptable salt or solvate thereof, on contact with aqueous media. The first composition is formulated so that it provides modified release of retigabine or a pharmaceutically acceptable salt or solvate thereof, on contact with aqueous media. In some embodiments, the second composition is arranged so that in use it releases at least 50% of the retigabine or a pharmaceutically acceptable salt or solvate thereof, in the stomach. In some embodiments, the second composition can be arranged so that in use it releases at least 60% of the retigabine or a pharmaceutically acceptable salt or solvate thereof, in the stomach. In some embodiments, the second composition can be arranged so that in use it releases at least 75% of the retigabine or a pharmaceutically acceptable salt or solvate thereof, in the stomach.

In some embodiments, the first composition can arranged so that in use it releases at least 90% of the retigabine or a pharmaceutically acceptable salt or solvate thereof in the intestines. In some embodiments, the first composition can be arranged so that in use it releases at least 95% of retigabine or a pharmaceutically acceptable salt or solvate thereof in the intestines. In some embodiments, the dosage form is a tablet.

During human trials of an embodiment of the oral dosage form of the invention it was found that, release of the drug is such that the mean maximum plasma level concentration (“Cₘₐₓ”) value of the drug is maintained substantially independent of food during use, i.e. the observed Cₘₐₓ value is similar in both fasted and fed states during use. Accordingly, in one aspect the oral dosage form can be arranged to release retigabine or a pharmaceutically acceptable salt or solvate thereof, such that the mean maximum plasma level concentration (“Cₘₐₓ”) value of the drug is maintained substantially independent of food during use, i.e. no dose dumping occurred in the fed states during use.

In addition it has also been found that the oral dosage form releases the drug such that the mean area under the plasma concentration versus time curve over the dosing interval at steady state (“AUC”) observed on administration is maintained substantially independent of food during use, i.e. the observed AUC is similar in both fasted and fed states during use. Accordingly in one aspect, the oral dosage form can be arranged to release retigabine or a pharmaceutically acceptable salt or solvate thereof, such that the mean area under the plasma concentration versus time curve over the dosing interval at steady state (“AUC”) is maintained substantially independent of food during use, i.e. the observed AUC is similar in both fasted and fed states during use.

Thus, in a further aspect, in operation the oral dosage form releases retigabine or a pharmaceutically acceptable salt or solvate thereof, so that both the Cₘₐₓ value and AUC observed on administration are maintained substantially independent of food during use, i.e. no dose dumping occurs in the fed states during use. The compositions can be formed in any shape or mutual conformation providing the required objective of the invention is met.

The above reference to a core being erodable includes a situation where the core disintegrates partially or wholly, or dissolves, or becomes porous, on contact with the relevant environmental fluid so as to allow the fluid to contact the active agent. In some embodiments, the core disintegrates partially, while in other embodiments the core disintegrates wholly. In some embodiments, the core dissolves and in some embodiments, the core becomes porous.

In some embodiments, the invention provides an erosion of a coating in a pH dependent manner, while the core can release retigabine or a pharmaceutically acceptable salt or solvate thereof, by eroding in a non-pH dependent manner.

Although retigabine or a pharmaceutically acceptable salt or solvate thereof is more soluble in the stomach than the intestines, the core can be formulated so as to release drug to substantially the same extent in both the stomach and the intestines, i.e. the core is formulated to compensate for the pH dependency of retigabine.

The above reference to the coating being erodable includes the situation where the coating disintegrates partially or wholly, or dissolves, or becomes porous, on contact with an environmental fluid so as to allow the fluid to contact the core. In some embodiments, the coating can disintegrate partially. In some embodiments, the coating disintegrates wholly. In some embodiments, the coating dissolves. In some embodiments, the coating becomes porous. In some embodiments, the erodable coating is an enteric coating, i.e. it has a defined, pre-determined pH threshold at which it dissolves. In some embodiments, the coating erodes at pH greater than 4.5. In some embodiments, the coating erodes in the pH range from 4.5 to 8. In some embodiments, the coating erodes in the pH range 5 to 7. In some embodiments, the enteric coating is non-permeable.

Materials and their blends suitable for use as a pH-dependent erodable coating material in this invention include various polymethacrylate polymers, co-processed polyvinylacetate phthalate, cellulose acetate trimellitate, cellulose acetate phthalate, shellac, hydroxyropylmethylcellulose phthalate polymers and their copolymers and hypromellose acetate succinate. In some embodiments, the coating material is selected from cellulose acetate trimellitate (CAT), polyvinyl acetate phthalate, hydroxypropylmethylcellulose phthalate 50, hydroxpropylmethylcellulose phthalate 55, Acryl-eze™, Aquateric™, cellulose acetate phthalate, Eudragit™ L30 D, Eudragit™ L, Eudragit™ S and shellac. In particular embodiments, the coating material is Eudragit™ L30 D.

When necessary, the erodable coating can be modified by addition of plasticizers or anti-tack agents. Suitable materials for this purpose include waxy materials such as glycerides, for example glyceryl monostearate, or mono-/di-glycerides.

Typical sizes for the opening(s), when circular, to be formed in the coating are in the range 0.9 mm-6 mm of diameter, such as 1, 2, 3, 4 or 5 mms in diameter, depending on the overall size of the tablet and the desired rate of release. In one aspect of the invention the openings are circular with diameters of 2 or 4 mms. In another aspect of the invention the openings are circular with diameters of 3, 4 or 5 mm. The opening(s) can have any convenient geometrical shape, but a rounded shape, e.g. substantially circular or elliptical, is generally sufficient. More elaborate shapes, such as text characters or graphics, can also be formed, provided that the release rate can be made uniform in individual dosage forms. Typical sizes of non-circular openings are equivalent in area to the above mentioned sizes for circular openings, thus in the range of from about 0.6 to about 30 mm2.

For the purposes of the present invention, the term “opening” is synonymous with hole, aperture, orifice, passageway, outlet etc. The opening(s) may be formed by methods disclosed in U.S. Pat. No. 5,004,614. Typically opening(s) may be formed by drilling, for example using mechanical drill bits or laser beams, or by punches that remove the cut area. The formation of the opening(s) may by default remove a small portion of the exposed core. It is also possible to purposely form a cavity below the aperture as a release rate controlling device, the cavity exposing a greater initial surface area of core than a flat surface. Suitably, the opening(s) extend through the entire erodable coating such that there is immediate exposure of the core to the environmental fluid when the device is placed in the desired environment of use.

Also it is possible to form the opening(s) in situ when the dosage form is administered, by forming a coating containing pore-forming agents i.e. material that will dissolve in the stomach to create pores in the coating. Typically the pore forming agent is erodable in the pH range from 1 to 3.

In U.S. Pat. No. 5,004,614, the opening(s) includes about 10-60% of the total face area of the tablet i.e. the upper and lower surfaces of a biconvex tablet. In the present invention, the opening(s) may comprise 0.18 to 20%, such as 1 to 20% of the total face area.

Alternatively, it can be useful to characterize the rate controlling effect of the opening(s) by reference to the area of the opening(s) relative to the total surface area of the coated tablet. Additionally, especially in cases where the core erodes by undercutting of the edges of the opening(s), the rate controlling effect may be related to the total circumference of the opening(s).

A particularly unexpected finding is that two openings, for example one on each primary surface of a biconvex tablet, release an active agent from the core at a rate marginally greater than that of a single opening of the same overall area. It is also indicated that the variability of the release rate from the two openings is less than the variability of release rate from the corresponding single opening. Accordingly, in one embodiment of the invention, the coating of the core is provided with two or more openings. In some embodiments, the erodable coating surrounding the core is provided with two or more openings extending substantially completely through the coating but not penetrating the core and communicating from the environment of use to said core.

Where more than one opening is provided, the openings can be located on the same surface of the oral dosage form, or on different surfaces. In some embodiments, the oral dosage form has two openings, for example one on each of opposing surfaces. In some embodiments, the oral dosage form is a tablet having two opposed primary surfaces, each surface having one opening through the coating, and, in some embodiments, substantially completely through the coating. The core can be arranged so that one opening provides access to the first composition and the another opening provides access to the second composition.

As a protection for the core material, to prevent contamination via the opening(s) before dosing, it can be desirable to provide a conventional seal coating to either the core, or to the dosage form after formation of the opening(s). The seal coat may be a sub-coat or over-coat to the erodable coating. Additionally it can be desirable to provide a color coating to either the core, or to the dosage form after formation of the opening(s). The seal coat may be a sub-coat or over-coat to the erodable coating.

A further aspect of the present invention is a process for preparing an oral dosage form that includes (i) an erodable core, which core including a first modified release composition that includes retigabine or a pharmaceutically acceptable salt or solvate thereof, and a pharmaceutically acceptable carrier therefore; and (ii) an erodable coating around the core, which coating comprises one or more openings extending substantially completely through the coating but not penetrating the core and communicating from the environment of use to the core, wherein release of retigabine or a pharmaceutically acceptable salt or solvate thereof, from the erodable core occurs substantially through the opening(s) and through erosion of said erodable coating under pre-determined pH conditions which process includes the steps of i) formulating retigabine or a pharmaceutically acceptable salt or solvate thereof into a core; ii) coating the core with an erodable coating; and iii) creating one or more openings in the coating, the openings extending substantially completely through the coating but not penetrating the core and communicating from the environment of use to the core.

According to yet a further aspect of the present invention, there is provided a process for preparing an oral dosage form which dosage form includes a first modified release composition and a second composition, each composition including retigabine or a pharmaceutically acceptable salt or solvate thereof (“the drug”) and a pharmaceutically acceptable carrier therefor, wherein the first and second compositions are arranged to release drug at differing release rates on administration such that the rate of release of the drug from the dosage form is substantially independent of pH; which process includes the steps of sequentially or simultaneously: (i) formulating the drug into the first composition; and (ii) formulating the drug into the second composition; (iii) coating the core with an erodable coating; and (iv) creating one or more openings in the coating, the openings extending substantially completely through the coating but not penetrating the core and communicating from the environment of use to the core.

The first and second compositions can be prepared by compressing suitable ingredients in conventional manner to form a compacted mass in multiple layers, which includes the core of the dosage form (also referred to herein as “tablet core”). The tablet core can be prepared using conventional tablet excipients and formulation compression methods. Thus, the core typically includes the active agent or agents along with excipients that impart satisfactory processing and compression characteristics such as diluents, binders and lubricants. Additional excipients that can form part of the core of the device include disintegrants, flavorants, colorants, release modifying agents and/or solubilizing agents such as surfactants, pH modifiers and complexation vehicles. Typically, the active agent and excipients are thoroughly mixed prior to compression into a solid core. The core of the device can be formed by wet granulation methods, dry granulation methods or by direct compression. The core can be produced according to any desired pre-selected shape such as bi-convex, hemi-spherical, near hemi-spherical, round, oval, generally ellipsoidal, oblong, generally cylindrical or polyhedral, e.g. a triangular prism shape. The term “near hemi-spherical” is intended to be construed in the manner described in U.S. Pat. No. 5,004,614. In some embodiments, the core is formulated into a bi-convex shape, e.g. having two domed opposite surfaces.

Suitable materials for the first composition are a rate controlling polymer or matrix forming polymer, such as the drug delivery matrices described herein above. For example high molecular weight hypromellose (HPMC) 2910 (also known as E) or 2208 (also known as K), methylcellulose, polyethylene oxide, hydroxypropyl cellulose, xanthan gum, guar gum Eudragit NM, Eudragit NE, Kollidon SR, galactomannans, dextran, ethylcellulose, carbomer, carbopol, polycarbophil, sodium carboxymethylcellulose, hydroxyethylcellulose, hydroxyethylmethylcellulose, shellac, zein, cellulose acetate or combinations thereof.

In one aspect of the invention high molecular weight hypromellose 2910 (also known as E) or 2208 (also known as K) may be used. In some embodiments, the rate controlling polymer for the first composition is high molecular weight hypromellose 2208 (also known as K). If only one composition is present in the core, the rate controlling polymer for the first composition can be high molecular weight hypromellose 2910 (also known as E). In one aspect of the invention the rate controlling polymer or matrix forming polymer comprises 10-30% by tablet weight.

Additionally a lubricant can be added for example magnesium stearate, sodium stearyl fumarate, talc or stearic acid.

Suitable materials for the second composition, include saccharoses, for example lactose and maltose; mannitol, xylitol, calcium lactate, calcium silicate, dicalcium phosphate, trehalose or microcrystalline cellulose for example Avicel™. Additionally disintergrants or superdisinitergrants such as croscarmellose sodium or sodium starch glycolate, surfactants such as sodium lauryl sulphate can be added. In some embodiments, the second composition is predominantly mannitol. In some embodiments, the second composition includes as excipients, mannitol and magnesium stearate.

In a further aspect the retigabine can be wet granulated with other ingredients, for example mannitol, Avicel™ or HPMC to prepare granules which are then blended with other ingredients to form the first and second compositions which are then compressed as discussed above. The wet granulation process can be performed in a fluid bed processor, where the dry powder is fluidized by incoming air through the bottom of the equipment and the binder solution is sprayed into the fluidized powder. The wet powder is dried to the appropriate moisture level. Alternatively, the wet granulation process can be performed in a high shear mixer or an extruder or similar unit setup for continuous wet granulation. In one aspect of the invention the same granule can be used in both composition or alternatively separate granules could be prepared for each composition.

In a further aspect of the invention the retigabine can be micronized. Formulations containing micronized retigabine can allow administration of formulations containing lower drug quantities. They can provide more consistent pharmacokinetic profiles, and may allow a reduction in dosing regimen.

Fluid energy milling or micronization is a frequently used process for size reducing pharmaceutical materials. The parent Active Pharmaceutical Ingredient (API) is fed into the milling chamber at a feed rate which is defined in the batch record and set up at the start of each batch. This product feed rate is monitored at intervals throughout the run. The injection gas creates a reduced pressure zone at the venturi, and the powder is pulled into the milling chamber.

The mill has a number of nozzles which are evenly spaced along the interior wall of the milling chamber to create the necessary momentum for collisions to reduce the size of the input API. The milling chamber acts as a particle classifier by keeping larger particles inside the chamber through inertia and allowing smaller particles to escape with the gas into the collection bag through the internal classifier. Size reduction is achieved primarily through particle-particle collisions.

The core can be coated with a suitable pH dependent erodable material by any pharmaceutically acceptable coating method. Examples include coating methods disclosed in U.S. Pat. No. 5,004,614 and film coating, sugar coating, spray coating, dip coating, compression coating, electrostatic coating. Typical methods include spraying the coating onto the tablet core in a rotating pan coater or in a fluidised bed coater until the desired coating thickness is achieved. Suitably the coating is provided to add about 4 to 8 mg/cm2 or 5-7 mg/cm2 of dry polymer around the tablet surface area. Typically this results in an increase in weight (relative to the core) of from 3-10% or 5-10% by weight. Suitably, the coating has a thickness in the range 0.05 to 0.5 mm.

As indicated above, the oral dosage form of the present invention can be considered to be suitable for twice or once daily administration and during use is indicated to provide a therapeutic effect over an extended period of time, such as up to 24 hours, for example, up to 12, 14, 16, 18, 20 and 24 hours, per unit dose.

In a further aspect of the invention the oral dosage form provides sustained release retigabine or a pharmaceutically acceptable salt or solvate thereof, for example providing release of the active agent over a time period of up to 26 hours; suitably in the range of about 4 to about 24 hours; including in the range of about 4 to about 15 and for example about 4 to about 12 hours.

In yet a further aspect of the invention the oral dosage form provides pulsed release of retigabine or a pharmaceutically acceptable salt or solvate thereof, for example providing up to about 4, for example 2, pulses of active agent per 24 hours.

The quantity of retigabine or a pharmaceutically acceptable salt or solvate thereof to be used in accordance with the present invention is a matter to be determined based upon typical pharmaceutical considerations, e.g. known dosages for retigabine or a pharmaceutically acceptable salt or solvate thereof, and is not limited by the process of this invention.

In particular, where retigabine a pharmaceutically salt or solvate thereof is used in accordance with the present invention, a suitable dosage range can include up to 1500 mg, for example, 10 to 1500 mg, for example 20 to 800 mg, suitably 100 to 800 mg. Thus, suitable oral dosage forms of the invention comprise 40, 80, 160, 200, 320, 400, 480 or 640 mg of retigabine or a pharmaceutically acceptable salt or solvate thereof.

The amount of retigabine or a pharmaceutically acceptable salt or solvate thereof present in the first composition and the second composition may be varied in accordance with the desired dissolution profile.

In one aspect of the invention the first composition comprises 2 to 3 times as much retigabine as the second composition, suitable 2 to 2.5 times. For example, where the oral dosage form includes about 480 mg of retigabine or a pharmaceutically acceptable salt or solvate thereof, the tablet core suitably can include a layer comprising about 340 mg of retigabine or a pharmaceutically salt or solvate thereof, and a layer including about 140 mg of retigabine or a pharmaceutically salt or solvate thereof.

By adjustment of the release rates of the first and second compositions, and adjusting the other variables mentioned above and the surface area of the exposed core, the release rates in the different environmental conditions can be harmonized to obtain comparable release rates under different body environments, and so achieve more constant dosing to a patient.

Dissolution rates can be assessed by in vitro testing in solutions of the appropriate pHs. For example, when comparing dissolution in the stomach and intestines, tests can be carried out initially at about pH 1.3 with a transfer to about pH 6.4 after about 2 hours or about 4 hours, as an assumed time for residence in the stomach before emptying into the intestines of a notional patient in respectively fasted and fed conditions.

As mentioned above, retigabine or a pharmaceutically acceptable salt or solvate thereof when administered in an oral dosage form of this invention is indicated to be useful for the treatment and/or prophylaxis of the disorders describe above. In one aspect of the invention, retigabine or a pharmaceutically acceptable salt or solvate thereof when administered in an oral dosage form of this invention is indicated to be useful for the treatment and/or prophylaxis of epilepsy.

In one embodiment the present invention provides a method for the treatment and/or prophylaxis of the disorders described above; the method includes administering an oral dosage form in accordance with various embodiments disclosed herein that include retigabine or a pharmaceutically acceptable salt or solvate thereof, to a human or non-human mammal.

In some embodiments, the present invention provides a method for the treatment and/or prophylaxis of epilepsy. The method includes administering an oral dosage form described herein that includes retigabine or a pharmaceutically acceptable salt or solvate thereof, to a human or non-human mammal.

In a further embodiment the present invention provides an oral dosage form as described herein that includes retigabine or a pharmaceutically acceptable salt or solvate thereof for use in the treatment and/or prophylaxis of any of the above described diseases or disorders.

In a further embodiment, the present invention provides an oral dosage form of the invention including retigabine or a pharmaceutically acceptable salt or solvate thereof for use in the treatment and/or prophylaxis of the epilepsy.

It is understood that modifications which do not substantially affect the activity of the various embodiments of this invention are also included within the definition of the invention provided herein. Accordingly, the following examples are intended to illustrate but not limit the present invention.

### Example I

### Components and Proportions of Modified Release Formulations

This Example describes components and proportions of components for the formulation of compounds of formula I.

Table 1 provides ingredients and proportions of ingredients for formulation of pharmaceutical compositions into a modified release dosage form. For all the following Examples the proportion of active ingredient utilized ranges from 35% to 65% of the total dosage form with the remainder constituting binders, disintegrants, surfactants, release modifying agents, glidants or lubricants in ranges as shown in Table 1. The dry blend for direct compression or wet granulation of a portion of the formulation or wet granulation of entire formulation were used to manufacture granules and tablets.

TABLE 1
Exemplary Retigabine Modified Released (MR) formulations.

| | Range | |
|---|---|---|
| | (% of final | |
| Component | formulation) | Function |
| Retigabine | 35-65 | Active Pharmaceutical |
| | | Ingredient |
| Hypromellose 2208 | 1-30 | Drug delivery matrix |
| (Methocel K4M) | | |
| Dicalcium Phosphate | 0-10 | Filler/Binder |
| Microcrystalline Cellulose | 5-40 | Binder, Release Modifying |
| (Avicel PH-101) | | Agent |
| Hypromellose 2910 | 0-10 | Binder, Release Modifying |
| | | Agent |
| Copovidone | 0-10 | Binder |
| Polyethylene Glycol (PEG | 0-10 | Binder, Release Modifying |
| 6000, PEG8000) | | Agent |
| Crospovidone | 0-5 | Disintegrant |
| Croscarmellose Sodium | 0-5 | Disintegrant |
| Sodium Dodecyl Sulfate | 0-7 | Surfactant |
| Sucroester | 0-5 | Surfactant |
| Magnesium Stearate | 0-2 | Lubricant |
| Colloidal Silicone Dioxide | 0-2 | Glidant |

### Example II

### Preparation of Modified Release Formulations

This Example describes the methods of preparing the modified release formulations of the present invention and provides the components and respective proportions utilized in preparation of modified release formulations of the invention.

Methods described herein will be understood by the skills since many such methods are known in the art. Table 2 shows ingredients and proportions utilized in preparing several embodiments of the claimed invention. It is to be understood that the amounts and proportion of components used in Tables 1 and 2 may be apportioned into smaller or larger amounts, while maintaining the ingredient ratios, to produce the different modified release formulations of the invention. It should be further understood that such an apportionment of ingredients is also within the scope of the claims and the present invention.

Modified release formulations A, B, C, D, F and H were prepared as follows. Briefly, retigabine was milled and blended with microcrystalline cellulose, hypromellose 2208, crospovidone, and sodium dodecyl sulfate (SDS) in the proportions set out in Table 2 for 15 minutes. Caplets were prepared by tablet compression and completed with an enteric coating.

Modified release formulation E was prepared as follows. Retigabine was milled and blended with hypromellose 2208, copolyvidone, and granulated with a water solution of hypromellose 2910 in the fluid bed drier at a maximum temperature of 50° C. The granulation was blended with croscarmellose sodium and lubricated. Tablets were compressed and enteric coated.

Modified release formulation G was prepared as follows. Milled retigabine was mixed in a Robot Coupe high shear mixer with microcrystalline cellulose, hypromellose 2208, plasdone, and sodium dodecyl sulfate. While mixing at 1500 rpm binding solution was added. The wet granulation mass was passed through a sieve. The granulation was dried in an oven at 45° C. and subsequently blended with lubricant and croscarmellose sodium followed by compression into tablets.

Modified release formulation I was prepared as follows. Briefly, milled retigabine was mixed with a portion of microcrystalline cellulose and sucroester and granulated with water solution and hypromellose 2910 in the fluid bed drier at a maximum temperature of 50° C. The granulation was blended with hypromellose 2208, crospovidone and the remaining amount of the microcrystalline cellulose, lubricated and compressed in caplet shaped tablets.

TABLE 2
Ingredient proportions utilized in preparation of several modified
release formulations of the invention.

| | Modified Release Formulation | | | | | | | | |
|---|---|---|---|---|---|---|---|---|---|
| Substance (mg) | A | B | C | D | E | F | G | H | I |
| Retigabine | 200.0 | 200.0 | 200.0 | 200.0 | 200.0 | 200.0 | 200.0 | 200.0 | 200.0 |
| | (58) | (52.6) | (50.0) | (49.1) | (51.0) | (49.3) | | (52.5) | (53.0) |
| Hypromellose | 17.5 | 38.0 | 48.0 | 48.0 | 48.0 | 48.0 | 48.0 | 38.0 | 49.0 |
| 2208 | (5.1) | (10.0) | (12.0) | (12.0) | (12.2) | (11.8) | | (10.0) | (13.0) |
| Microcrystalline | 103.0 | 99.6 | 107.6 | 107.6 | 103.0 | 103.0 | 103.0 | 103.0 | 98.0 |
| Cellulose | (29.8) | (26.2) | (26.9) | (26.9) | (26.2) | (25.4) | | (27.0) | (26.0) |
| Hypromellose | | | | | 5.0 | 5.0 | 5.0 | 5.0 | 7.5 |
| 2910 | | | | | (1.3) | (1.2) | | (1.3) | (2.0) |
| Copolyvidone | | 15.2 | 16.0 | 16.0 | 16.0 | 16.0 | 16.0 | 13.0 | |
| | | (4.0) | (4.0) | (4.0) | (4.1) | (3.9) | | (3.4) | |
| Crospovidone | 7.0 | 3.8 | 4.0 | | 4.0 | 4.0 | 4.0 | 4.0 | 3.8 |
| | (2.1) | (1.0) | (1.0) | | (1.0) | (0.98) | | (1.0) | (1.0) |
| Croscarmellose | | | | | 16.0 | 12.0 | 12.0 | | |
| Sodium | | | | | (4.1) | (3.0) | | | |
| Sodium | 17.5 | 17.5 | 17.6 | 17.6 | | 18.0 | 18.0 | 18.0 | |
| Dodecyl Sulfate | (5.1) | (4.6) | (4.4) | (4.4) | | (4.4) | | (4.7) | |
| Sucroester | | | | | | | | | 18.9 |
| | | | | | | | | | (5.0) |
| Dicalcium | | | | 18.4 | | | | | |
| Phosphate | | | | (4.6) | | | | | |
| Total Tablet | 345 | 380 | 400 | 400 | 392 | 406 | 406 | 381 | 377 |
| Weight | | | | | | | | | |
| Acryl-eze ® | 29.3 | 32.3 | 34.0 | 34.0 | 33.3 | 34.5 | 34.0 | 32.4 | |
| | (8.5) | (8.5) | (8.5) | (8.5) | (8.5) | (8.5) | (8.5) | (8.5) | |

The modified release formulations of Table 2 were tested for dissolution characteristics, at pH 7.5 and pH 2.0, to determine the anticipated extent of dissolution in the stomach as well as in the gastrointestinal tract (GI tract). To make the determination, the rate of retigabine release into solution using USP dissolution apparatus, was determined for each of the modified release formulations of Table 2. In vitro dissolution studies were carried out using a buffered media similar to procedures employed in USP compendial dissolution testing. USP Type II apparatus, pH 7.5 buffer and 1.7% (w/v) SDS or simulated gastric juice (0.1N HCl) were employed to dissolve and measure percent of drug released over the stated time period (see, for example, U.S. Pharmacopeia, 28th revision, Chapter 711, second supplement, (Aug. 1, 2005 to Dec. 31, 2005). Results are reported as % (w/w) of retigabine released as a function of time.

Table 3 shows the rate of retigabine release over 4 hours for modified release formulations A-I. All formulations demonstrated varying dissolution character in pH 7.5 borate buffer containing SDS. “A” demonstrated rapid dissolution with complete dissolution occurring within 0.5 hours. The release rate of “B” was measured at 46% with 100% percent of retigabine released after 3 hours. Modified release formulation “C” yielded a 23% rate of release at 0.5 hours and 84% retigabine release after 4 hours. The rate of release for modified release formulation “D” was relatively rapid yielding 75% rate of release at 0.5 hours and 100% release occurring at 2 hours. The rate of release of formulation “E” was not determined. Rate of release of formulation “F” was 40% at 0.5 hours with 94% released at the 4 hour time point. The percent release of formulation “G” was 28% at 0.5 hours and measured at 90% at 4 hours. Formulation “H” demonstrated a relatively slow rate of release with 14% of retigabine release occurring at 0.5 hours and 72% at 4 hours. Modified release formulation “I” was tested both in pH 7.5 buffered media and 0.1N HCl. In buffered media, modified release formulation “I” yielded a relatively low release rate with 8% retigabine release occurring at 0.5 hours and 66% in 4 hours. In 0.1N HCl, the rate of retigabine release at 0.5 hours was 11% and 34% at the 2 hour time point.

Because release rates were variable the modified release formulations of the present invention also allow varying degrees of systemic exposure in patients requiring unique treatments.

TABLE 3
Release Rates during dissolution of several modified release
formulations of the invention over a 4 hour time period.

| Modified | | | | | | |
|---|---|---|---|---|---|---|
| release | Dissolution | Percent Rate of Release (Hours) | | | | |
| formulation | Media | 0.5 | 1 | 2 | 3 | 4 |
| A | pH 7.5 | 100.0 | | | | |
| | buffer with | | | | | |
| | 1.7% SDS | | | | | |
| B | pH 7.5 | 46.0 | 70.0 | 95.0 | 100.0 | |
| | buffer with | | | | | |
| | 1.7% SDS | | | | | |
| C | pH 7.5 | 23.0 | 37.0 | 55.0 | 71.0 | 84.0 |
| | buffer with | | | | | |
| | 1.7% SDS | | | | | |
| D | pH 7.5 | 75.0 | 95.0 | 100.0 | | |
| | buffer with | | | | | |
| | 1.7% SDS | | | | | |
| E | pH 7.5 | ND | ND | ND | ND | ND |
| | buffer with | | | | | |
| | 1.7% SDS | | | | | |
| F | pH 7.5 | 40.0 | 50.0 | 65.0 | 80.0 | 94.0 |
| | buffer with | | | | | |
| | 1.7% SDS | | | | | |
| G | pH 7.5 | 28.0 | 42.0 | 65.0 | 75.0 | 90.0 |
| | buffer with | | | | | |
| | 1.7% SDS | | | | | |
| H | pH 7.5 | 14.0 | 22.0 | 39.0 | 57.0 | 72.0 |
| | buffer with | | | | | |
| | 1.7% SDS | | | | | |
| I | pH 7.5 | 8.0 | 18.0 | 37.02 | 53.0 | 66.0 |
| | buffer with | | | | | |
| | 1.7% SDS | | | | | |
| J | 0.1N HCl | 11.0 | 20.0 | 34.0 | | |

### Example III

### Preparation of Modified Release Formulations with Differing Amounts of Ingredients

This Example describes compositions and proportions of several modified release formulations of the invention containing 200 mg of retigabine.

Several modified release formulations were prepared employing 200 mg of retigabine and varying proportions of ingredients of the invention. Table 4 provides several modified release formulations of 200 mg of retigabine. The ratio of ingredients per milligram of tablet is provided in parenthesis. For Formulation 9, extra granular SDS was used to prepare the composition. It is to be understood that one skilled in the art may employ a larger or smaller apportionment of ingredients, as described in Table 4, while maintaining the ratio of ingredients, to produce a comparable modified release formulation. It is further to be understood that such an apportionment falls within the scope of the present invention.

The modified release formulations were prepared as described in Example II above.

TABLE 4
Modified release formulations of the invention.
Amounts are provided in mg/tablet.
Numbers shown in parenthesis provide the percentage
of each component in a formulation.

| | Formulation ID | | | | | | | | |
|---|---|---|---|---|---|---|---|---|---|
| Ingredients | 1 | 2 | 3 | 4 | 5 | 6 | 7 | 8 | 9 |
| Retigabine | 200.0 | 200.0 | 200.0 | 200.0 | 200.0 | 200.0 | 200.0 | 200.0 | 200 |
| | (57.1) | (52.6) | (50.1) | (52.6) | (57.1) | (52.6) | (51.4) | (49.1) | (50.5) |
| Hypromellose | 17.5 | 38.0 | 48.0 | 21.0 | | 47.5 | 48.0 | 48.0 | 48.0 |
| 2208 (Methocel | (5.0) | (10.0) | (12.0) | (5.5) | | (12.5) | (12.3) | (11.8) | (12.1) |
| K4M CR) | | | | | | | | | |
| Microcrystalline | 103.0 | 99.6 | 107.5 | 106.1 | 101.1 | 98.0 | 102.6 | 102.7 | 102.6 |
| Cellulose | (29.4) | (26.2) | (26.9) | (27.9) | (28.9) | (25.8) | (26.3) | (25.2) | (25.9) |
| (Avicel PH 101) | | | | | | | | | |
| Hypromellose | | | | | 10.4 | 10.4 | 5.0 | 5.0 | 5.0 |
| 2910 | | | | | (3.0) | (2.7) | (1.3) | (1.2) | (1.26) |
| Copovidone | | 15.2 | 16.0 | 11.4 | | | 16.0 | 16.0 | 16.0 |
| | | (4.0) | (4.0) | (3.0) | | | (4.1) | (3.9) | (4.0) |
| Crospovidone | 7.0 | 3.8 | 4.0 | | | 3.8 | 4.0 | 4.0 | 4.0 |
| | (2.0) | (1.0) | (1.0) | | | (1.0) | (1.0) | (0.98) | (1.0) |
| Croscarmellose | | | | | 17.5 | 17.5 | 9.0 | 9.0 | 16.0 |
| sodium | | | | | (5.0) | (5.0) | (2.3) | (2.2) | (4.0) |
| Sodium Dodecyl | 17.5 | 17.5 | 17.5 | | | | | 17.5 | |
| Sulfate | (5.0) | (4.6) | (4.4) | | | | | (4.3) | |
| Sucroester | | | | 17.5 | 17.5 | 17.5 | | | |
| (Ryoto-Sugar- | | | | (4.6) | (5.0) | (4.6) | | | |
| Ester S-1670) | | | | | | | | | |
| Dicalcium | | | | 17.5 | | | | | |
| phosphate | | | | (4.6) | | | | | |
| Magnesium | 3.0 | 1.9 | 3.0 | 3.5 | 3.5 | 2.8 | 2.8 | 2.8 | 2.8 |
| Stearate | (0.9) | (0.5) | (0.7) | (0.9) | (1.0) | (0.75) | (0.7) | (0.7) | (0.7) |
| Silicon Dioxide | 2.0 | 4.0 | 4.0 | 3.0 | | | 2.0 | 2.0 | 2.0 |
| | (0.6) | (1.1) | (1.0) | (0.8) | | | (0.5) | (0.5) | (0.5) |
| Uncoated tablet | 350.0 | 380.0 | 400.0 | 380.0 | 350.0 | 380.0 | 389.4 | 407.0 | 396.4 |
| weight (mg) | | | | | | | | | |
| Acryl-Eze (8.5%) | 29.8 | 32.3 | 34.0 | 32.3 | 29.8 | | 33.1 | 34.6 | 33.7 |

### Example IV

### Statistical Analysis of Pharmacokinetic Parameters of Several Modified Release Formulations

This Example provides a comparison of plasma retigabine pharmacokinetic parameters in fed and fasted subjects dosed with 400 mg retigabine modified release formulations.

In order to more formally assess the plasma concentration time profile for modified release formulations containing retigabine, PK studies were conducted in fed and fasted subjects over an 72-hour time period. In total, fourteen subjects received single oral doses of the formulations.

In one study, formulations 1, 3, 5, and 6 containing 400 mg of retigabine were dosed orally in fed or fasted subjects and the results shown in Table 5 below. In general, subjects were weighed and orally administered retigabine-containing modified release formulations. Fed subjects were dosed with food. Fasted subjects were fed 4 hours post dose and fasted overnight pre-dose. Blood was collected by venipuncture and plasma isolated by centrifugation. Plasma was frozen at −80° C. until time of analysis. Retigabine concentrations were determined by validated methods. Samples were analyzed in a standard reference standard concentration range that was linear throughout the range of concentrations.

The area under the curve (AUC) values (ng-hr/mL) values were determined using standard non-compartmental methods and least squares (LS) means, mean ratio (relative to a 200 mg dose of immediate release tablets) and 90% confidence interval of the mean ratio are provided in Table 5. Table 5 shows that all modified release formulations tested yielded comparable LS-means AUC values. Consistent with the administration of a 400 mg MR formulation dose, and a 200 mg IR formulation dose, the mean ratios of AUC values for all modified release formulations ranged from 144.48 to 235.7 (MR 5, 2×200 mg, fasted). In addition, a food effect was observed for some formulations with increased AUC values measured in fed subjects versus fasted. However, some formulations did not show a food effect.

TABLE 5
Statistical analysis of pharmacokinetic parameters for plasma
retigabine following administration of a single oral dose of 400 mg
retigabine sr formulations versus 200 mg retigabine
immediate release (IR) formulation are shown.

| | | Pharmacokinetic | |
|---|---|---|---|
| Period | | Parameters | % MR (90% CI)* |
| 2 | 400 mg SR | Cₘₐₓ | 53.24 (41.59, |
| | (Formulation 1) Fasted | | 68.15) |
| | Versus 200 mg IR | C₁₂ₕ | 231.25 (185.25, |
| | Fasted | | 288.68) |
| | | AUC₀₋₂₄ | 136.87 (114.92, |
| | | | 163.00) |
| | | AUC₀₋ₜ | 137.15 (116.63, |
| | | | 161.28) |
| | | Ae₀₋₂₄ | 120.95 (97.94, |
| | | | 149.38) |
| 3 | 400 mg SR | Cₘₐₓ | 46.61 (36.41, |
| | (Formulation 6) Fasted | | 59.66) |
| | Versus 200 mg IR | C₁₂ₕ | 181.54 (145.42, |
| | Fasted | | 226.62) |
| | | AUC₀₋₂₄ | 121.75 (102.23, |
| | | | 144.99) |
| | | AUC₀₋ₜ | 121.93 (103.69, |
| | | | 143.38) |
| | | Ae₀₋₂₄ | 94.17 (76.25, |
| | | | 116.30) |
| 4 | 400 mg SR | Cₘₐₓ | 74.71 (58.06, |
| | (Formulation 5) Fasted | | 96.12) |
| | Versus 200 mg IR | C₁₂ₕ | 259.62 (206.98, |
| | Fasted | | 325.64) |
| | | AUC₀₋₂₄ | 161.01 (134.70, |
| | | | 192.48) |
| | | AUC₀₋ₜ | 225.41 (191.04, |
| | | | 265.95) |
| | | AUC_{0-inf} | 179.44 (157.68, |
| | | | 204.19) |
| | | Ae₀₋₂₄ | 170.08 (137.06, |
| | | | 211.05) |
| 5 | 400 mg SR | Cₘₐₓ | 86.23 (67.02, |
| | (Formulation 1) Fed | | 110.95) |
| | Versus 200 mg IR | C₁₂ₕ | 363.49 (289.80, |
| | Fasted | | 455.93) |
| | | AUC₀₋₂₄ | 178.55 (149.37, |
| | | | 213.44) |
| | | AUC₀₋ₜ | 299.83 (254.12, |
| | | | 353.76) |
| | | AUC_{0-inf} | 235.70 (207.13, |
| | | | 268.22) |
| | | Ae₀₋₂₄ | 156.04 (125.75, |
| | | | 193.63) |
| 6 | 400 mg SR | Cₘₐₓ | 38.76 (30.12, |
| | (Formulation 1) Fasted | | 49.87) |
| | Versus 200 mg IR | C₁₂ₕ | 198.30 (158.10, |
| | Fasted | | 248.73) |
| | | AUC₀₋₂₄ | 103.28 (86.40, |
| | | | 123.46) |
| | | AUC₀₋ₜ | 180.89 (153.31, |
| | | | 213.43) |
| | | AUC_{0-inf} | 144.48 (126.96, |
| | | | 164.41) |
| | | Ae₀₋₂₄ | 120.04 (96.73, |
| | | | 148.96) |
| 7 | 400 mg SR | Cₘₐₓ | 44.62 (34.25, |
| | (Formulation 3) Fasted | | 58.13) |
| | Versus 200 mg IR | C₁₂ₕ | 177.79 (140.14, |
| | Fasted | | 225.54) |
| | | AUC₀₋₂₄ | 106.78 (88.53, |
| | | | 128.78) |
| | | AUC₀₋ₜ | 235.02 (197.58, |
| | | | 279.55) |
| | | AUC_{0-inf} | 207.97 (180.84, |
| | | | 239.18) |
| | | Ae₀₋₂₄ | 99.19 (79.05, |
| | | | 124.44) |
| 8 | 400 mg SR | Cₘₐₓ | 60.30 (46.28, |
| | (Formulation 3) Fed | | 78.55) |
| | Versus 200 mg IR | C₁₂ₕ | 306.81 (241.85, |
| | Fasted | | 389.22) |
| | | AUC₀₋₂₄ | 140.17 (116.22, |
| | | | 169.05) |
| | | AUC₀₋ₜ | 270.98 (227.81, |
| | | | 322.32) |
| | | AUC_{0-inf} | 213.74 (186.60, |
| | | | 244.82) |
| | | Ae₀₋₂₄ | 105.65 (84.20, |
| | | | 132.55) |

*= 90% CI and % Mean Ratios (% MR) were calculated based on ln-transformed parameters

FIG. 3 shows a comparison of the pharmacokinetic profiles (PK; mean values) of Formulations 1, 3, 5, and 6 in subjects dosed orally in either a fasted or fed state compared to an immediate release control.

Absorption and elimination profiles (mean values measured over an 72 hour time period) for the modified release formulations 1, 3 and 6 were relatively similar with a plateau-like concentration profile maintained for approximately 15 to 20 hours. Although concentrations were higher for formulations 1 and 3 when dosed with food, plateau-like concentration profiles were still maintained for 12-20 hours. Formulation 3 provided similar total exposure whether dosed with or without food. Overall, formulations 1, 3 and 6 demonstrated plateau-like concentration profiles that resulted in concentrations being maintained near the level of peak concentrations for 12-20 hours, substantially longer than would have been expected based on in vitro dissolution results.

A separate PK study was conducted with formulations 8 and 9 as summarized in Table 6 below.

TABLE 6
Statistics of pharmacokinetic parameters of retigabine in healthy male and
female subjects following administration of a single oral dose of 200 mg of
treatments T1, T2 and R are shown.

| | Treatment T1 | Treatment T2 | Treatment R |
|---|---|---|---|
| Parameter | Mean ± SD | Mean ± SD | Mean ± SD |
| AUC(0-inf) | 2840.68 ± 1001.98 | 2385.47 ± 914.29 | 3503.57 ± 1002.84 |
| (ng · hr/mL) | 2631.93 (35.27)* | 2191.49 (38.33)* | 3359.69 (28.62)* |
| Cₘₐₓ(ng/mL) | 120.79 ± 45.15 | 93.71 ± 31.81 | 451.46 ± 180.17 |
| | 112.75 (37.38)* | 88.78 (33.94)* | 410.18 (39.91)* |
| Tₘₐₓ(hr) | 10.00 (6.00, 24.05)** | 11.02 (4.00, 36.00)** | 1.00 (0.50, 4.00)** |
| T_{lag}(hr) | 1.00 (0.00, 4.00)** | 1.00 (0.00, 3.00)** | 0.00 (0.00, 0.00)** |

number of subjects = 34 for each treatment regimen,
*Geometric mean (% CV),
**Median (Range),
Treatment T1 retigabine 1 × 200 mg MR Formulation 8,
Treatment T2 retigabine 1 × 200 mg MR Formulation 9,
Treatment R retigabine 2 × 100 mg immediate release (IR).

### Example V

### Dissolution Profiles of Modified Release Retigabine Formulations 1-9

This Example provides dissolution rates and profiles of retigabine formulated utilizing formulations 1-9.

Using methods described in Example II, Formulations 1-9 were dissolved utilizing USP compendial dissolution procedures. The rate of retigabine release in 0.1N HCl (simulated in vivo conditions of exposure to gastric juice) for 1 hour, followed by 4-5 hours in pH 7.5 borate buffer was measured over a 4-6 hour period. FIG. 4 provides the release profile. There was little dissolution of any of Formulations 1-5 and 7-9 in 0.1 N HCl (pH 2.0) while the immediate release (IR) retigabine formulation fully dissolves in this media in a 1 hour time period as shown.

Overall, these studies indicate that modified release formulations of the present invention allow for maintenance of dosage form integrity in the presence of a low pH environment (pH-2.0) as occurs in the stomach. The formulations also allow for modified and controlled dissolution of retigabine in higher pH environments such as occurs in the GI tract.

### Example VII

### Solubility of Retigabine in Aqueous Solution

This Example provides the solubility character of retigabine with varying pH values.

In order to assess the solubility of retigabine in varying pH environments, solubility studies using retigabine as an exemplary active ingredient were conducted in aqueous solution at 37° C. A representative solubility curve for retigabine is shown in FIG. 5. The results indicate that maximum solubility was observed at pH 1.5 with solubility at approximately 16 mg/ml in aqueous solution. Increasing to pH 2.0 resulted in a solubility of just under 4 mg/ml. Increasing to pH 3.0 resulted in nearly complete insolubility under aqueous conditions. Solubility was low in pH ranges of between pH 4.0 to pH 12.0. The pH profile indicates that retigabine would be expected to dissolve in the stomach under acidic (e.g. pH 2.0) conditions, although this would be prevented by the presence of an enteric coating.

### Example VIII

### 480 mg Tablet with a 2 mm Aperture and Example IX: 480 mg Tablet with a 4 mm Aperture

In the following examples the granules for Layers 1 and 2 were prepared separately using standard wet granulation procedures. The granules were then blended with the remaining ingredients for each layer and compressed. The two layers were then compressed together, film coated using standard procedures to add the color coat and then the enteric film coat was applied by spraying the coating onto the tablet core in a rotating pan coater. Apertures of 2 mm or 4 mm were mechanically drilled in the enteric coat producing tablets of Examples VIII and IX respectively.

TABLE 7

mg/tab

Layer 1, granulation

| | |
|---|---|
| Retigabine | 339.4 |
| Avicel PH101 | 77.4 |
| HPMC 603 | 16.3 |
| Total | 433.1 |

Layer 2, granulation

| | |
|---|---|
| Retigabine | 145.4 |
| Mannitol, 160C | 27.3 |
| HPMC 603 | 9.1 |
| Total | 181.8 |

Layer 1, Blend

| | |
|---|---|
| Layer 1 granules | 433.1 |
| HPMC, K100LV | 139.4 |
| Mannitol, 200SD | 71 |
| Mg Stearate | 6.1 |
| Total | 649.6 |

Layer 2, Blend

| | |
|---|---|
| Layer 2 granules | 181.8 |
| Mannitol, 200SD | 57.1 |
| Mg Stearate | 1.5 |
| Total | 204.4 |

Aqueous Film Coat

| | |
|---|---|
| Opadry, orange | 22 |
| Total | 912.0 |

Enteric Film Coat

| | |
|---|---|
| Eudragit L30-D55 (dry basis) | 22.69 |
| Triethyl citrate | 2.38 |
| Mono-/di-glycerides | 0.65 |
| Polysorbate 80 | 0.28 |
| Totals | 938.0 |

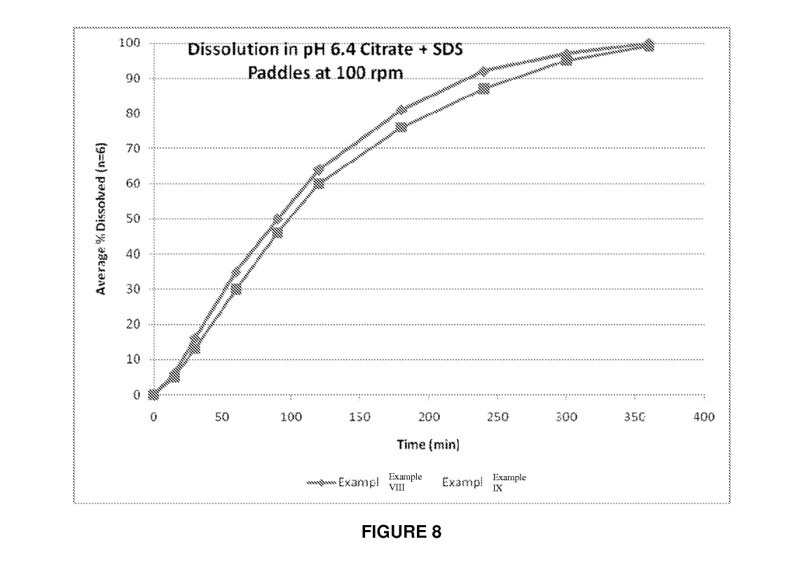

Dissolution profiles for the dosage forms of Examples VIII and IX are shown in FIG. 8 of the accompanying drawings. The dissolution method used is as follows:

Dissolution is determined in accordance with USP General Chapter <711>. The procedure uses USP Apparatus 2 with a paddle speed of 100 rpm. The medium is 20 mM sodium citrate with 2.0% w/v sodium dodecyl sulphate, pH 6.4 (900 mL at 37 C). The amount of dissolved retigabine is quantitated by UV spectroscopy using external standards.

Retigabine Common granules are prepared by wet granulation. The granules are manufactured by fluidizing the retigabine and microcrystalline cellulose powder and spraying the hypromellose in solution onto the fluidized bed. After adding the appropriate amount of hypromellose, the wet granules are dried to an appropriate moisture level and milled to the desired particle size

TABLE 8

| Description | % w/w |
|---|---|
| Retigabine | 78.4 |
| Microcrystalline Cellulose | 17.9 |
| Hypromellose 603 | 3.7 |
| Total | 100 |

These common granules are then used to prepare Layers 1 and 2 by mixing with the other ingredients and the layers are prepared by compression, are aqueous film coated, then the enteric coat is applied. Apertures are mechanically drilled.

TABLE 9

| | | | | |
|---|---|---|---|---|
| | Ex. X | Ex. XI | Ex. XII | Ex. XIII |
| | 160 | 320 | 480 | 640 |
| Description | mg/tab | mg/tab | mg/tab | mg/tab |

Layer 1, blend

| | | | | |
|---|---|---|---|---|
| Retigabine Common granule | 142.9 | 285.8 | 428.7 | 571.6 |
| Hypromellose, K100LV | 72.3 | 134.7 | 138.0 | 164.0 |
| Mannitol | 11.4 | 22.8 | 33.7 | 44.8 |
| Microcrystalline cellulose | 11.4 | 22.8 | 33.7 | 44.8 |
| Mg Stearate | 2.0 | 3.9 | 5.9 | 7.8 |
| Layer 1, Totals | 240.0 | 470.0 | 640.0 | 833.0 |

Layer 2, Blend

| | | | | |
|---|---|---|---|---|
| Retigabine Common granule | 61.2 | 122.5 | 183.7 | 245.0 |
| Mannitol | 13.3 | 26.5 | 30.0 | 40.0 |
| Microcrystalline cellulose | 5.0 | 10.0 | 14.3 | 19.3 |
| Mg Stearate | 0.5 | 1.0 | 2.0 | 2.7 |
| Layer 2, Totals | 80.0 | 160.0 | 230.0 | 307.0 |
| Total Tablet weight | 320.0 | 630.0 | 870.0 | 1140.0 |

Aqueous Film Coat

| | | | | |
|---|---|---|---|---|
| Opadry, orange | 13.0 | 19.0 | 23.0 | 27.0 |
| Totals | 333.0 | 649.0 | 893.0 | 1167.0 |

Enteric Film Coat

| | | | | |
|---|---|---|---|---|
| Eudrgit L30 D55 (dry basis) | 14.84 | 22.69 | 27.93 | 33.16 |
| Triethyl citrate | 1.55 | 2.38 | 2.93 | 3.48 |
| Mono-/di-glycerides | 0.43 | 0.65 | 0.80 | 0.95 |
| Polysorbate 80 | 0.18 | 0.28 | 0.34 | 0.41 |
| totals | 350.0 | 675.0 | 925.0 | 1205.0 |
| Drilled Aperture Size (mm) | 2.5 mm | 3 mm | 4 mm | 5 mm |

To investigate the bioavailability of five MR formulations [Example VIII, IX and Formulation 8 (Table 4) and two reference MR formulations] administered in the fed and fasted state as a single dose relative to administration of the immediate release (IR), in the fed and fasted state.

To investigate the effect of food on the MR formulations administered as a single dose. This was an open-label, randomized single dose, cross-over phase I study conducted in healthy volunteers. Subjects were assigned to either a fasted dosing regimen (Group 1) or to a fed dosing regimen (Group 2 or Group 3) (high-fat meal) with a washout of 5-7 days between cross-over sessions. All meals were standardized. Each of the modified release formulations was administered in either the fasted or fed state.

Subjects in Part A, Group 1 received each of the MR formulations and the IR formulation in a randomized 6-way crossover fashion in the fasted state.

TABLE 10
Dosing Regimens for Group 1 (Fasted)
Group 1 (fasted)

400 mg IR (A)
480 mg MR Formulation 1- Example IX
480 mg MR Formulation 2- Example VIII
480 mg Formulation 3- Formulation 8 (Table 4)
480 mg MR Formulation 4- Reference Formulation
480 mg MR Formulation 5- Reference Formulation

Subjects in Group 2 received the IR formulation and the MR formulations in a randomized manner (4-way crossover) and subjects in Group 3 received IR formulation and 2 MR formulations in a randomized manner (3-way crossover) with a high fat meal (see Table 11)

TABLE 11
Dosing Regimens for Group 2 and Group 3(Fed)

| | Part A, Group 3 |
|---|---|
| Part A, Group 2 (high fat meal) | (high fat meal) |
| 400 mg IR (G) | 400 mg IR |
| 480 mg MR Formulation 1- Example IX | 480 mg MR Formulation 4- |
| 480 mg MR Formulation 2- Example VIII | Reference Formulation |
| 480 mg MR Formulation 3- Formulation 8 | 480 mg MR Formulation 5- |
| (Table 4) | Reference Formulation |

Subjects were healthy adult male and female volunteers between 18 and 65 years of age (inclusive). For Group 1, approximately 20 male subjects were to be enrolled such that approximately 16 subjects completed dosing and pharmacokinetic assessments. For Group 2 and Group 3, approximately 12 male subjects were to be enrolled into each group such that approximately 10 subjects completed dosing and pharmacokinetic assessments.

Pharmacokinetics (AUC and Cₘₐₓ) for immediate release and modified release formulations were the primary pharmacokinetic parameters. The secondary pharmacokinetic parameters were tₘₐₓ and t_{1/2} of immediate release and modified release formulations. Plasma samples for retigabine pharmacokinetic analysis were obtained prior to dosing and up to 72 hours post dose on each dosing occasion. Plasma concentrations for pharmacokinetic analysis of retigabine were determined by validated assay methodologies.

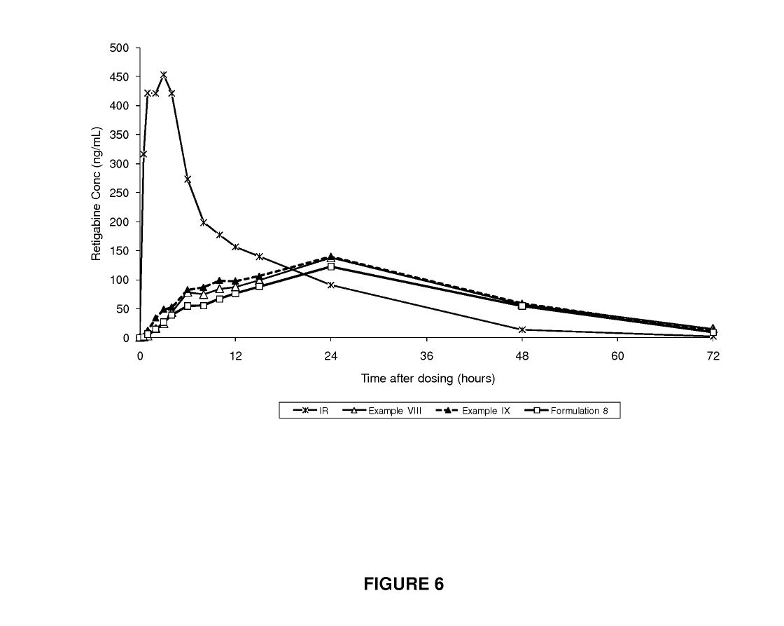

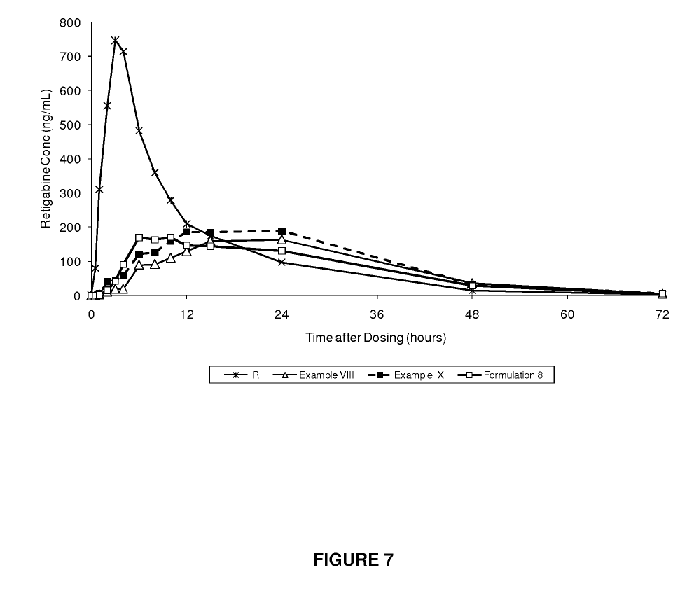

Mean retigabine concentration-time profiles following administration of retigabine IR and retigabine MR (Example VIII, Example IX and Formulation 8 of Table 4) in the fasted state are shown in FIG. 6 and in the fed state (high fat meal) in FIG. 7. For the MR formulations it was not possible to accurately determine the half-life, therefore AUC(O-t) was used as the primary endpoint. The PK results for this modified release formulation in the fasted and fed state are provided in Table 12. CVb % indicates the percent between subject coefficient of variation.

TABLE 12
Summary of Dose Normalized PK parameters following administration
of IR tablets (400 mg) and Modified Release tablets Example VIII and
Example IX (480 mg) in Fasted State (geomean (CVb %)) (Group 1)

| | | | **AUC(0-t) |
|---|---|---|---|
| | *Tmax (h) | **Cmax (ng/mL) | (ng · h/mL) |
| IR (n = 18) | 2 (0.5-4) | 620 (28%) | 5720 (28%) |
| Formulation 8 (Table 4) | 24 (4-48) | 120 (48%) | 3754 (49%) |
| Example VIII (n = 18) | 24 (10-48) | 138 (40%) | 4670 (34%) |
| Example IX (n = 19) | 24 (10-48) | 140 (29%) | 4640 (29%) |

*Median (range),
**Normalized to 400 mg.

TABLE 13
Summary of Dose Normalized PK parameters following administration
of IR Tablets (400 mg) and modified release tablets Example VIII and
Example IX (480 mg) in the Fed State (geomean (CVb %)) (Group 2)

| | | **Cmax | **AUC(0-t) |
|---|---|---|---|
| | *Tmax (h) | (ng/mL) | (ng · h/mL) |
| IR (n = 14) | 2.5 (0.5-4) | 840 (34%) | 6350 (24%) |
| Formulation 8 (Table 4) | 10 (4-48) | 210 (41%) | 4617 (25%) |
| Example VIII (n = 10) | 19.5 (6-24) | 189 (33%) | 4720 (25%) |
| Example IX (n = 11) | 12 (6-24) | 236 (29%) | 5630 (21%) |

*Median (range),
**Normalized to 400 mg

In both the fasted and fed state administration of Examples VIII, Example IX and Formulation 8 of Table 4 resulted in a reduction of Cₘₐₓ to between approximately 20% and 30% of that observed for IR. For both Example VIII and Example IX in the fasted state, Tₘₐₓ occurred between 10 and 48 hours post dose compared to between 0.5 and 4 hours for retigabine IR. Tₘₐₓ for Formulation 8 of Table 4 occurred between 4 and 48 h. For both Example VIII and Example IX in the fed state, Tₘₐₓ occurred between 6 and 24 hours post-dose compared to between 0.5 and 4 hours for retigabine. Tₘₐₓ for Formulation 8 occurred between 4 and 48 h.

Throughout this application various publications have been referenced. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific examples and studies detailed above are only illustrative of the invention. It should be understood that various modifications can be made without departing from the spirit of the invention. Accordingly, the invention is limited only by the following claims.

What is claimed is:

1. A modified release pharmaceutical formulation, comprising:
about 30-70% N-(2-amino-4-(fluorobenzylamino)-phenyl) carbamic acid ethyl ester (retigabine), or a pharmaceutically acceptable salt, solvate or hydrate thereof;
about 5-30% of a drug delivery matrix comprising hydroxypropylmethylcellulose (HPMC); and
an enteric polymer,
said pharmaceutical formulation producing a sustained plasma concentration of said retigabine following administration to a subject for 4-20 hours longer than the time required for in vitro release of 80% of said retigabine.

2. The formulation of claim 1, further comprising an anionic surfactant selected from sodium dodecyl sulfate and sodium lauryl sulfate.

3. The formulation of claim 1, wherein the enteric polymer is selected from polyvinylacetate phthalate, hydroxypropylmethylcellulose acetate succinate (HPMC-AS), and a copolymer of two or more of methyl methacrylate, methacrylic acid, ethyl acrylate, and methyl acrylate.

4. The formulation of claim 1, further comprising about 5-40% of a modified release polymer/binder.

5. The formulation of claim 4, wherein said modified release polymer/binder comprises microcrystalline cellulose.

6. The formulation of claim 5, wherein the modified release polymer/binder further comprises hydroxypropylmethylcellulose.

7. The formulation of claim 5, wherein the binder further comprises copovidone.

8. The formulation of claim 1, further comprising about 0.5-10% of a disintegrant.

9. The formulation of claim 8, where said disintegrant comprises crospovidone.

10. The formulation of claim 9, wherein said disintegrant further comprises croscarmellose sodium.

11. The formulation of claim 1, further comprising a lubricant.

12. The formulation of claim 11, wherein said lubricant comprises magnesium stearate.

13. The formulation of claim 1, further comprising a glidant.

14. The formulation of claim 13, wherein said glidant comprises silicon dioxide.

15. The formulation of claim 1, wherein retigabine is administered in a dose ranging from about 5 mg to about 800 mg.

16. The formulation of claim 15, wherein retigabine is administered in a dose ranging from about 400 mg to about 700 mg.

17. A method of treating a disorder characterized by nervous system hyperexcitability comprising administering to a subject in need thereof an effective amount of a pharmaceutical formulation according to claim 1.

18. The method of claim 17, wherein said disorder characterized by nervous system hyperexcitability comprises a seizure disorder.

19. The method of claim 17, wherein said administration produces an anti-seizure, muscle relaxing, fever reducing, peripherally analgesic or anti-convulsive effect.

20. The method of claim 17, wherein said disorder characterized by nervous system hyperexcitability further comprises a disorder characterized by activation of voltage-gated potassium channels.

21. The method of claim 17, wherein said administration produces an increase in the channel opening probability of KCNQ2/3 channels or in neuronal M currents.
